(19) United States
(12) Patent Application Publication
(10) Pub. No.: US 2004/0110252 A1
(43) Pub. Date: Jun. 10, 2004

(54) Yeast-based assay

(76) Inventor: John Davey, West Midlands (GB)

Correspondence Address: Paul D Greeley, Ohlandt Greeley Ruggiero & Perle, 10th Floor, One Landmark Square, Stamford, CT, 06901-2682, US

(21) Appl. No.: 10/450,097

(22) PCT Filed: Dec. 10, 2001
(86) PCT No.: PCT/GB01/05460

(30) Foreign Application Priority Data
Dec. 8, 2000 (GB) 0030038.4

(51) Int. Cl. C12P 21/06; C12N 1/18; C07K 14/705

(52) U.S. Cl. 435/69.1; 435/254.2; 435/320.1; 530/350

(57) ABSTRACT

The application discloses Sz. pombe yeast cells which have been modified so that a F-Protein Coupled Receptor (GPCR)-regulated signalling pathway is derepressed during the mitotic phase of cell growth. Isolated nucleic acid molecules encoding constructs used to make the yeast cells, uses of the cells and nucleic acid molecules to study GPCR pathways and to isolate compounds which effect in such pathways are also disclosed.

The application relates to modified yeast cells which may be used to study the activity of G-protein coupled receptors (GPCRs). The yeast cells used are Schizosaccharomyces pombe (Sz. pombe) containing a reporter gene-promoter construct. The invention also relates to isolated nucleic acid encoding the reporter gene-promoter construct and to uses of the yeast cells and nucleic acid molecules in assays.

GPCRs are an important class of receptors in all eukaryotic organisms, including mammals and yeast, and are responsible for conveying hormonal and sensory signals to the cell machinery (reviewed in Baldwin, 1994). Such receptors have a common structure comprising 7-transmembrane domains with an extracellular N-terminus and C-terminal cytoplasmic tail. GPCRs are usually coupled to a heterotrimeric G protein composed of Gα, Gβ and Gγ subunits. Binding of a ligand to the receptor stimulates a change in the G protein where guanosine diphosphate (GDP) bound to the Gα subunit is exchanged for guanosine triphosphate (GTP). Accompanying conformational changes result in the dissociation of Gα-GTP from the Gβγ dimer, either of which can modulate the activity of effector proteins to bring about changes in cell behaviour.

GPCRs control the physiology of all major organ systems and have been important targets for therapeutic and diagnostic advances, providing clinically successful drugs in nearly all the major pharmaceutical markets. Many of the 200 or so well characterised GPCRs are associated with at least one drug and about 60% of commercially available drugs act on GPCRs, providing some $27 billion in annual sales world-wide. There are another 100 or so GPCRs for which ligands have not yet been identified. These so called ‘orphan' receptors are likely to include many that will become important drug targets. Analysis of the human genome indicates that there are probably another 500 orphan GPCRs that will need to be characterised. There is therefore considerable interest in developing drug leads targeted at the GPCRs.

One approach to the identification of new drugs is the development of high throughput screens (HTS) for GPCRs. In most cases, the target GPCR is expressed in a host system such that activation of the receptor leads to a change in cell behaviour. Screening can then identify drug leads that either mimic the action of the natural ligand (agonists) or block the receptor (antagonists). All eukaryotic cells contain GPCRs and each can be adapted for HTS but it is not always practical to do this and most screens use a limited range of host systems. These include mammalian cells, frog melanocytes, insect cells and yeast Each system has its advantages and disadvantages. For example, mammalian cells might seem the obvious choice for studying human GPCRs but they are difficult and expensive to work with and screens are often complicated by the inherent presence of receptors closely related to the GPCR being studied. The presence of related receptors can also complicate screens involving frog melanocytes and insect cells. These problems do not apply to yeast and many have turned to using this relatively simple cell as a surrogate host for screening human GPCRs.

G-protein coupled receptors are known in yeast. Accordingly, yeast, such as Saccharomyces cerevisiae (S. cerevisiae) have been used to study GPCR-regulated signalling systems. Yeast cells are particularly advantageous because they have the ability to be easily manipulated, at low cost and with high levels of production. Unlike bacteria, yeast has the potential to perform eukaryotic post-translational modifications that may affect receptor function (Reiländer and Weib, 1998). The mechanism of transcriptional activation in yeast and higher eukaryotes may be very similar. For example, yeast upstream activation sites (UAS) and some transcriptional activators have been found to have very similar activity to that of their mammalian equivalents (Jones et al., 1988).

Most work carried out on yeast systems has been on S. cerevisiae. There are many reports describing the coupling of exogenous GPCRs to the intracellular signalling machinery in S. cerevisiae. These include the human β₂-adrenergic (King et al., 1990), rat somatostatin (Price et al., 1995; Bass et al., 1996), rat adenosine A_{2A} (Price et al., 1996), human growth hormone releasing hormone (Kajkowski et al., 1997), human lysophosphatidic acid (Erickson et al., 1998), human formyl peptide receptor like-1 (Klein et al., 1998), human C5a chemoattractant (Klein et al., 1998; Baranski et al., 1999), mushroom pheromone (Olesnicky et al., 1999), human somatostatin SST₂ (Brown et al., 2000), human somatostatin SST₅ (Brown et al., 2000), human serotonin 5-HT_{1A} (Brown et al., 2000), human serotonin 5-HT_{1D} (Brown et al., 2000), human melatonin ML_{1B} (Brown et al., 2000), human purinergic P2Y₁ (Brown et al., 2000), human purinergic P2Y₂ (Brown et al., 2000), human adenosine A_{2B} (Brown et al., 2000), human UDP-glucose (Chambers et al., 2000), human protease-activated receptor (Swift et al., 2000), human muscarinic M₁ (Erlenbach et al., 2001), human muscarinic M₃ Erlenbach et al., 2001), human muscarinic M₅ (Erlenbach et al., 2001) and human vasopressin V₂ (Erlenbach et al., 2001).

However, not all GPCRs couple to the signalling machinery in S. cerevisiae. Receptors that fail to couple are not normally reported but as many as 40% of human GPCRs are not functional in S. cerevisiae.

The fission yeast Schizosaccharomyces pombe (Sz. pombe) is becoming popular as an alternative genetically tractable eukaryote which is not only phylogenetically distant from S. cerevisiae, but in several aspects of its cell and molecular biology seems to more closely resemble a higher eukaryotic cell (Reiländer and Weib, 1998; Allshire et al., 1987). Sz. pombe would therefore seem to provide an attractive alternative to the budding yeast. Unfortunately, all previously reported attempts to couple exogenous GPCRs to the signalling machinery in Sz. pombe have been unsuccessful. It appears that although the receptors are expressed they fail to couple to the intracellular signalling machinery in the yeast Examples include bacteriorhodopsin (Hildebrandt et al., 1993), human dopamine D_{2S} (Sander et al., 1994); human neurokinin NK2 (Arkinstall et al., 1995) and human β₂-adrenergic (Ficca et al., 1995).

This application describes how Sz. pombe may be manipulated, for example by way of modification, to allow the coupling of exogenous GPCRs to the intracellular signalling machinery and hence generate strains suitable for high throughput screening for agonists and antagonists that affect the activity of the exogenous receptors.

Fission yeasts, such as Sz. pombe, have two distinct growth cycles. Firstly, they have a normal vegetative or mitotic cycle in which haploid cells simply divide by fission. Secondly, they have a meiotic cycle. In the meiotic cycle, a yeast cell conjugates with a second yeast cell to form a diploid cell. The diploid cell then undergoes meiosis and sporulation to form four haploid spores. Such spores are very resilient and the meiotic cycle is usually triggered when environmental conditions for the yeast no longer support mitotic growth. For example, the meiotic cycle in Sz. pombe is usually triggered by nitrogen starvation.

Conjugation in Sz. pombe is controlled by the reciprocal action of diffusible mating pheromones. M cells (of mating type minus) release M-factor which prepares P cells (mating type plus) for mating, while P cells release P-factor which stimulates M cells for mating. Binding of the pheromones to their receptors on the surface of the target cell activates an intracellular signalling pathway which leads to changes in the pattern of gene transcription and prepares the cell for mating. Responses induced by the pheromones include G₁ arrest of the cell cycle, an increase in agglutination, and the elongation of the cell to form a shmoo. The M-receptor and P-receptor to which the M-factor and P-factor pheromones bind are examples of G-protein coupled receptors. On binding of the pheromone to the receptor, a Gα subunit is released. This has a positive role in signal transduction within the Sz. pombe cell, as indeed is the case in many mammalian cells. This contrasts with S. cerevisiae, in which the Gα subunit is a negative regulator. Accordingly, the Sz. pombe system can be thought to be more closely analogous to GPCRs in higher eukaryotes such as mammals.

It has now been realised that an appropriately modified Sz. pombe would be a good model for studying GPCRs for identifying components of GPCR pathways, for identifying mutants in the GPCR pathways and for identifying compounds which stimulate or inhibit GPCR-regulated signalling pathways.

Sz. pombe also appears to have greater cell wall permeability than S. cerevisiae. This may prove to be invaluable in the study of receptors with large or complex ligands.

A first aspect of the invention provides a Schizosaccharomyces pombe (Sz. pombe) yeast cell comprising:

(i) a G-Protein Coupled Receptor (GPCR)-regulated signalling pathway which is derepressed during the mitotic phase of cell growth;

(ii) a reporter system for reporting a signal mediated by the GPCR-regulated signalling pathway

and wherein:

(a) the GPCR is heterologous, and/or

(b) the reporter system comprises a reporter gene and a promoter, the reporter gene being operatively linked to the promoter, and the promoter being regulatable by the GPCR, at least one of the reporter genes and the promoter being heterologous.

Expression of some of the components of the GPCR-regulated signalling machinery is normally repressed in Sz. pombe during mitotic growth and it is necessary to remove this repression in order to study signalling during the mitotic phase of cell growth. Methods for derepressing the GPCR-regulated signalling machinery are discussed below.

It has been discovered that maintaining a derepressed GPCR-regulated signalling pathway during the mitotic phase of cell growth allows Sz. pombe to be used to study GPCRs. That is, the cell has one or more signalling components activated during the mitotic phase to enable, for example, the binding of a suitable ligand to the GPCR to increase or reduce transcription of a reporter gene.

The yeast cell will generally comprise one or more mutations to derepress the GPCR-regulated pathway in mitotic growth. A nutritional control pathway can be disrupted by mutation for this purpose.

It is normally necessary to starve Sz. pombe cells to induce them to mate and derepress the GPCR-regulated signalling pathway. The relatively high level of cytoplasmic cAMP that exists during mitotic growth is reduced as nutrients become limiting and this helps to trigger sexual development. Strains lacking adenylate cyclase (which converts ATP to cAMP) have no cytoplasmic cAMP but grow reasonably well. They are derepressed for sexual development and respond to mating pheromones during mitotic growth. Accordingly, preferably the yeast cell is adenylate cyclase deficient. More especially, the cyr1 gene, which encodes adenylate cyclase, is physically or functionally removed or disrupted, for example by insertion of a DNA sequence. The inserted DNA sequence may be anything convenient, but in a preferred embodiment of the invention the inserted DNA may comprise a reporter gene; the ura4 gene is one example, as will be discussed below. The cyr1 gene is discussed in detail in the article by John Davey and Olaf Nielsen (Davey and Nielsen, 1994). Other methods for bypassing the nutritional control of the signalling machinery are available and may be used to derepress the GPCR-regulated signalling pathway in cells of the invention. This could include mutation of any gene that has the effect of repressing sexual differentiation. Such genes include those encoding certain protein kinases repressing sexual differentiation, including the pat1 gene. The use of a mutation in this gene to bypass the nutritional control and derepress the GPCR-regulated signalling pathway has been described (Davey and Nielsen, 1994).

As indicated above, Davey and Nielsen, 1994 discloses the identification of mutants involved in sexual differentiation and pheromone response. A temperature-sensitive pat1 mutant (pat1-114) allows the arrest of mitotic growth in response to M-factor. A mutation in the adenylate cyclase gene (cyr1) was also studied. The authors indicated that cells containing such a mutation have a problem in that they become insensitive or adapted to the pheromone. The perceived problems identified by the authors of the paper, have, in contrast with the analogous situation in Saccharomyces cerevisiae, now been found not to present a practical difficulty in heterologous Sz. pombe systems: specifically, it has been found that Sz. pombe cells containing a mutant cyr1 and a heterologous GPCR or a suitable heterologous reporter gene do not have a serious problem with desensitisation.

The reporter system allows signal transduction to be measured in a variety of ways. For example, suitable reporter system includes the association or dissociation of signalling components (to include, for example, the association of proteins with stimulated GPCRs, the dissociation of Gα subunits from negative regulators such as the Gβγ subunits), the generation of second messengers (such as Ca²⁺ mobilisation, changes in cyclic AMP levels, GTP hydrolysis, phospholipid hydrolysis), the modification of signalling components (such as the phosphorylation of e.g. MAP kinases, MAP kinase kinases or MAP kinase kinase kinases) or altered transcription of a gene. Transcription of a gene can be measured directly (for example, mRNA expression may be detected by Northern blots) or indirectly (for example, the protein product may be measured by a characteristic stain or intrinsic activity).

Preferably the yeast comprises a nucleic acid molecule encoding a heterologous reporter gene, or an endogenous reporter gene, operatively linked to a promoter that is regulated by a GPCR-regulated signalling pathway. By operatively linked, we mean that the heterologous reporter gene, or the endogenous reporter gene, is linked to the promoter in such a way that the promoter is capable of directing transcription of the reporter gene.

The reporter gene may be any nucleic acid sequence encoding a detectable gene product. The gene product may be an untranslated RNA product such as mRNA or antisense RNA. Such untranslated RNA may be detected by techniques known in the art, such as PCR, Northern or Southern blots. Alternatively, the reporter gene may encode a polypeptide, such as protein or peptide, product. A polypeptide may be detected immunologically or by means of its biological activity. The reporter gene may be any known in the art. The reporter gene need not be a natural gene, and the term “gene” in this sense should not be taken to imply identity-with any natural gene. Reporter genes useful in the invention may be the same as certain natural genes, but may differ from them either in terms of non-coding sequences (for example one or more naturally occurring introns may be absent) or in terms of coding sequences.

The reporter gene may encode a protein that allows the yeast cell to be selected by, for example, a nutritional requirement. For example, the reporter gene may be the ura4 gene which encodes orotidine-5′-phosphate decarboxylase. The ura4 gene encodes an enzyme involved in the biosynthesis of uracil and offers both positive and negative selection. Only cells expressing ura4 are able to grow in the absence of uracil, where the appropriate yeast strain is used. Cells expressing ura4 die in the presence of 5-fluoro-orotic acid (FOA) as the ura4 gene product converts FOA into a toxic product. Cells not expressing ura4 can be maintained by adding uracil to the medium. The sensitivity of the selection process can be adjusted by using medium containing 6-azauracil, a competitive inhibitor of the ura4 gene product. The his3 gene (encodes imidazoleglycerol-phosphate dehydratase) is also suitable for use as a reporter gene that allows nutritional selection. Only cells expressing his3 are able to grow in the absence of histidine, where the appropriate yeast strain is used

The reporter gene may encode for a protein that allows the yeast to be used in a chromogenic assay. For example, the reporter may be the lacZ gene from Escherichia coli. This encodes the β-galactosidase enzyme. This catalyses the hydrolysis of β-galactoside sugars such as lactose. The enzymatic activity of the enzyme may be assayed with various specialised substrates, for example X-gal (5-bromo-4-chloro-3-indoyl-β-D-galactoside) or o-nitrophenyl-β-D-galactopyranoside, which allow reporter enzyme activity to be assayed using a spectrophotometer, fluorometer or a luminometer.

The gene encoding green fluorescent protein (GFP), which is known in the art, may also be used as a reporter gene.

The reporter gene may also encode a protein that is capable of inducing the cell, or an extract of a cell, to produce light. For example, the reporter gene may encode luciferase. The luciferase reporter genes are known in the art. They are usually derived from firefly (Photinous pyralis) or sea pansy (Renilla reniformis). The luciferase enzyme catalyses a reaction using D-luciferin and ATP in the presence of oxygen and Mg²⁺ resulting in light emission. The luciferase reaction is quantitated using a luminometer that measures light output. The assay may also include coenzyme A in the reaction that provides a longer, sustained light reaction with greater sensitivity.

An alternative form of enzyme that allows the production of light is aequorin, which is known in the art.

Most preferably, the reporter gene encodes β-lactamase. This reporter gene has certain advantages over, for example, lacZ. There is no background activity in mammalian cells or yeast cells, it is compact (29 kDa), it functions as a monomer (in comparison with lacZ which is a tetramer), and has good enzyme activity. This may use CCF2/AM, a FRET-based membrane permeable, intracellularly trapped fluorescent substrate. CCF2/AM has a 7-hydroxycoumarin linked to a fluorescein by a cephalosporin core. In the intact molecules, excitation of the coumarin results in efficient FRET to the fluorescein, resulting in green fluorescent Cleavage of the CCF2 by β-lactamase results in spatial separation of the two dyes, disrupting FRET and causing cells to change from green to blue when viewed using a fluorescent microscope. The retention of the cleaved product allows the blue colour to develop over time, giving a low detection limit of, for example, 50 enzyme molecules per cell. This results in the reporter gene being able to be assayed with high sensitivity. It also allows the ability to confirm results by visual inspection of the cells or the samples.

The nucleic acid molecule comprising the reporter gene under the control of the GPCR-regulated promoter may additionally comprise one or more additional regulatory elements, such as upstream activating sequences (UAS), termination sequences and/or secretory sequences known in the art. The secretory sequences may be used to ensure that the product of the reporter gene is secreted out of the yeast cell.

Preferably the promoter is regulatable by a yeast mating pheromone binding to its GPCR. The yeast mating pheromone may especially be P-factor pheromone. This is especially preferred because the P-factor pheromone is relatively easy to produce.

The promoter is preferably an endogenous Sz. pombe promoter which is regulated by the GPCR. However, it does not have to be endogenous. Certain heterologous promoters may be found to be so regulatable, or may be engineered to be, for example by inclusion of a TR-box motif as described by Aono, et al. (1994).

More preferably, the promoter is the sxa2 promoter, or a homologue or analogue thereof. By homologue or analogue we mean a promoter which may contain one or more changes to the nucleic acid sequence encoding the sxa2 promoter but which maintains the same functional activity as the sxa2 promoter. The sxa2 gene to which the sxa2 promoter is attached in wild-type cells, encodes a carboxypeptidase that, in wild-type cells, inactivates P-factor by removal of the C-terminal leucine residue (Ladds et al., 1996). Use of the sxa2 promoter for construction of a GPCR-regulated reporter is advantageous because the promoter is tightly regulated by the P-factor receptor (the GPCR) to which the P-factor pheromone binds. Only one copy of the sxa2 promoter exists in wild-type cells. Accordingly, it is possible to remove the naturally occurring sxa2 promoter and its associated sxa2 gene and replace it with a construct containing the reporter gene under the transcriptional control of the sxa2 promoter. This promoter-reporter construct may be integrated into the chromosome of the yeast cell.

Integrating the promoter-reporter gene construct into the chromosome of the yeast cell is advantageous because a known number of reporter genes are then found within each cell. If the promoter-reporter gene construct is placed on a plasmid, then the number of reporter genes in each cell may vary since the copy number of the plasmid may vary considerably and is not constant.

Inactivating the endogenous sxa2 gene, for example by at least partially deleting the sxa2 gene, can improve the sensitivity of the assay when P-factor is used to stimulate the GPCR. This is because inactivation of the carboxypeptidase reduces inactivation of the P-factor which may be used to stimulate the GPCR The reporter gene may be linked to any remaining sxa2 gene, for example to form a fusion protein. Alternatively, the entire sxa2 gene may be deleted and the reporter gene inserted in its place.

Preferably, the yeast cell used exhibits a stable mating type. Mating type in Sz. pombe is determined by information carried at the mat1 locus. Haploid cells containing the mat1-P segment, which contains the mat1-Pc and mat1-Pm genes, are ‘+’ (P or plus), and those with mat1-M, encoding mat1-Mc and mat1-Mm are ‘−’ (M or minus). Expression of mat1-Pc and mat1-Mc are required for expressing the genes that encode the pheromones and their receptors and hence establish the pheromone communication system. AU 4 mat1 genes are required for meiosis. There are two further mating loci, mat2 and mat3 where P and M information is stored but not expressed. In wild-type homothallic strains the information at mat2 and mat3 is frequently transferred to the mat1 locus and cells switch mating type approximately once every three generations. Cultures of such strains are therefore normally a mixture of both mating types (P and M). Even normal heterothallic strains are relatively unstable. Strains with a stable mating type can be generated by either deleting the mat2 and mat3 loci or by mutating the switching machinery, to produce a yeast cell exhibiting a stable mating phenotype (Davey, 1998).

Continued exposure to stimulus will lead to desensitisation of the signalling pathway. Several mechanisms are known to contribute to the desensitisation process. Selected mutations in the genes encoding proteins involved in desensitisation can lead to hypersensitivity and an inability to adapt to stimulation. This could be an advantage when using the strains in high throughput screens.

The yeast cell may be rgs1 deficient. Strains lacking rgs1 or having reduced Rgs1 (the product of the rgs1 gene) activity are hypersensitive to pheromone stimulation (Watson, et al., 1999).

The yeast cell may also be pmp1 deficient. The pmp1 gene encodes a dual specificity phosphatase that dephosphorylates the MAPK. Strains lacking this phosphatase exhibit an increased response following stimulation of the cells with a ligand for the GPCR.

The GPCR may be a naturally occurring yeast pheromone receptor. Alternatively, the receptor may be replaced, or contain in addition thereto, an heterologous receptor from another cell. When the GPCR is heterologous, it may be from any species other than Sz. pombe. The GPCR may be from a plant species or an animal species, particularly mammals, including economically significant non-human mammals. In one of the most important aspects of the invention, however, it will be a human GPCR. The GPCR may be any GPCR which it is desired to investigate by means of the invention. For example, the yeast cell may express an orphan receptor. That is, a receptor of unknown specific activity, but which has been identified by its homology to other GPCR receptors. The yeast cell may be modified to produce such orphan receptors using techniques known in the art. For example, a plasmid containing a nucleic acid sequence encoding for the orphan receptor operably linked to suitable promoter and regulatory sequences may be inserted into the yeast cell. The receptors may be modified to include a signal sequence that functions in Sz. pombe. Suitable signal sequences include those of Mam2, Map3 and of other gene products secreted by the Sz. pombe cells. If the wild-type heterologous GPCR cannot be made functional in Sz. pombe, it may be mutated for this purpose. In addition, the Sz. pombe cells may express endogenous GPCRs in a functional form.

The Sz. pombe cell must contain a G protein that is activated by the GPCR and can interact with the rest of the yeast intracellular signalling machinery. The endogenous Sz. pombe Gα subunit (Gpa1) may be able to couple the heterologous receptor to the intracellular signalling machinery. However, it may be necessary to engineer the Sz. pombe cell to produce a heterologous or chimeric G protein subunit (or subunits). At least 16 Gα subunits have been identified in mammals and a given GPCR usually activates only one or a small subset of Gα subunits. The amino- and carboxy-termini of Gα subunits do not share significant homology, but there are several generalisations that can be made. For example, the amino-termini have been implicated in association with Gβγ subunits and with membranes through N-terminal myristoylation. Interaction with the receptor is thought to involve the carboxy-termini as mutants lacking the 5 C-terminal residues of the Gα subunit fail to couple to their receptors (see, for example, Hirsch et al., 1991) and peptides based on the C-terminal region of the Gα subunit bind to receptors (Hamm et al., 1988; Palm et al., 1990; Rasenick et al., 1994). Work with chimeric Gα subunits further supports a critical role for the C-terminal residues in conferring receptor specificity (Voyno-Yasenetskaya et al., 1994; Liu et al., 1995). Thus, the A1 adenosine receptor naturally couples through Gi but can couple via a Gα chimera in which the C-terminal 4 residues of Gq were exchanged for those of Gi2 (Conklin et al., 1993) and the SST3 somatostatin receptor does not couple through Gs but can be coupled to adenylate cyclase by replacing the last S residues of Gs with those from Gi2 (Komatsuzaki et al., 1997).

Several reports have demonstrated that heterologously expressed GPCRs can couple to the intracellular signalling machinery in S. cerevisiae. Some of these receptors can interact with the endogenous Gα subunit (encoded by the GPA1 gene), including those for rat somatostatin (Price et al., 1995), rat A_{2A} adenosine (Price et al., 1996), human lysophosphatidic acid (Erickson et al., 1998) and human UDP-glucose (Chambers et al., 2000). Several other receptors, including that for human growth hormone releasing hormone, do not couple to the S. cerevisiae Gpa1 (Kajkowski et al., 1997). In order to attain coupling of these receptors to the intracellular signalling machinery, the S. cerevisiae Gα subunit can be replaced by a mammalian Gα subunit or by a chimeric Gα subunit in which the C-terminal region of the yeast Gα subunit is replaced with the equivalent region of the mammalian Gα subunit. Many examples of the use of chimeric Gα subunits are available (Price et al., 1995; Bass et al., 1996; Kajkowski et al., 1997; Klein et al., 1998; Baranski et al., 1999; Swift et al., 2000). In some instances, production of the chimeric Gα may involve the replacement of as few as S residues from thy C-terminus of the endogenous yeast Gα subunit with the equivalent residues from the mammalian Gα subunit. Such constructs are sometimes referred to as ‘Gα-transplants’. There are several reports describing the use of Gα-transplants in S. cerevisiae (Olesnicky et al., 1999; Brown et al., 2000; Chambers et al., 2000; Erlenbach et al., 2001).

The use of Gα-transplants based on the endogenous Sz. pombe Gα subunit may be used to improve the coupling of heterologous GPCRs. To ensure the correct stoichiometric relationship between the Gα-transplant and the Gβγ subunits, it may be necessary to replace the chromosomal copy of the natural Sz. pombe Gα gene (gpa1) with the equivalent construct encoding the Gα-transplant. However, it is also likely that expression from other promoters is compatible with coupling of the Gα subunits to the receptors.

Yeast cells of the invention containing the Gα-transplants, and vectors, such as plasmids, cosmids, etc. containing nucleic acid encoding the transplants are included in the invention.

The yeast cell may additionally comprise one or more nucleic acid molecules, such as plasmids, encoding for one or more peptides or proteins, to allow the peptide or protein to be assayed for its effect on GPCR-regulated activity of the reporter system Alternatively, one or more other chemical compounds may be added to determine the effect of the compound on reporter system activity.

Preferably, the yeast cells contain an auxotrophic marker that allows the selection of plasmids in the yeast cells. The leu1 mutation provides one such marker and makes growth of the cells dependent upon the addition of leucine or on the introduction of a plasmid containing the leu1 gene. Similar mutations can also be made to genes involved in the biogenesis of other nutrients (including histidine, lysine and arginine). Such markers include ade1, ade6, arg3, CAN1, his3, his7 and ura4, all of which are known in the art.

Plasmids containing the nucleic acid encoding for a peptide or protein to be assayed may contain one or more promoter, termination and processing signal sequences. Suitable promoters include the thiamine repressed nmt1 promoter. This is repressed by the presence of thiamine. Other suitable promoters include adh1 and fbp1, which are known in the art

The plasmid may also contain a yeast autonomous replication sequence (ARS) to enable the plasmid to replicate in the Sz. pombe cells.

A bacterial origin of replication (ori), together with one or more bacterial selection markers, such as the ampicillin or tetracycline-resistant genes, may also be included to allow the plasmid to be replicated within bacterial systems prior to insertion into yeast cells. Additionally, the plasmids may include one or more restriction endonuclease sites to enable nucleic acid sequences encoding the peptide or proteins of interest to be inserted. Most preferably, the nucleic acid sequence encoding the peptides or proteins is random and/or may be in the form of a conformational library. Such libraries are known in the art. This allows the production of random peptides to identify peptide regulators of interest. This also allows a library of yeast cells containing different peptides to be produced.

One or more nucleic acid sequences encoding for known peptides or proteins may be introduced into the cell. This allows, for example, a mammalian GPCR-regulated pathway to be reconstituted within a yeast cell.

The strain may additionally contain an ade6 mutation that helps to make diploid strains of Sz. pombe more stable. This is useful where diploid strains of yeast are desirable.

It is not intended that the modifications to Sz. pombe described above necessarily be the only modifications made to the cell. Further modifications can be made as required for tailoring the system to particular circumstances.

A further aspect of the invention provides an isolated nucleic acid molecule comprising a promoter regulatable by G-Protein Coupled Receptor (GPCR)-regulated signalling pathway in Schizosaccharomyces pombe, operatively linked to a reporter gene. It is preferred that the promoter be an sxa2 promoter or homologue or analogue thereof operatively linked to a reporter gene. The sxa2 promoter and/or reporter genes may be as previously described.

For the avoidance of doubt, in this context, by reporter gene we mean any detectable gene which is not a naturally occurring sxa2 gene.

A further aspect of the invention provides the use of a Schizosaccharomyces pombe (Sz. pombe) yeast cell comprising:

(i) a G-Protein Coupled Receptor (GPCR)-regulated signaling pathway which is derepressed during the mitotic phase of cell growth;

(ii) a reporter system for reporting a signal mediated by the GPCR-regulated signalling pathway; or

(iii) an isolated nucleic acid molecule as defined above to study GPCR-regulated activity.

A further aspect of the invention provides an assay comprising the use of a Schizosaccharomyces pombe (Sz. pombe) yeast cell comprising:

(i) a G-Protein Coupled Receptor (GPCR)-regulated signalling pathway which is derepressed during the mitotic phase of cell growth;

(ii) a reporter system for reporting a signal mediated by the GPCR-regulated signalling pathway; or

(iii) isolated DNA molecule as defined above.

The invention also provides a method of determining the effect of a compound on GPCR-regulated activity comprised in the steps of:

(i) providing a Schizosaccharomyces pombe (Sz. pombe) yeast cell comprising:

(a) a G-Protein Coupled Receptor (GPCR)-regulated signalling pathway which is derepressed during the mitotic phase of cell growth;

(b) a reporter system for reporting a signal mediated by the GPCR-regulated signalling pathway;

(ii) introducing the compound to the yeast cell; and

(iii) noting the output of the reporter system, for example by determining an amount of reporter gene product produced by the yeast cell.

The amount of reporter gene product or other reporter system output may be compared with a control yeast without the compound.

The invention also relates to the use of a Schizosaccharomyces pombe (Sz. pombe) yeast cell comprising:

(a) a G-Protein Coupled Receptor (GPCR)-regulated signalling pathway which is derepressed during the mitotic phase of cell growth;

(b) a reporter system for reporting a signal mediated by the GPCR-regulated signalling pathway;

to identify a compound which acts as the receptor. The compound may be the or a natural ligand for the receptor or be an agonist or antagonist (or partial agonist or partial antagonist). Such compounds affect the ability of the receptor to regulate the GPCR-regulated signalling pathway. The invention therefore encompasses the use of such a yeast cell containing an orphan GPCR to identify compounds that affect the ability of the orphan receptor to regulate the promoter is also provided.

The yeast cell, as defined above, may be used to identify a regulator or a mutant of a GPCR-regulated pathway.

The invention also provides a method of identifying a reagent that modulates GPCR-regulated signalling, comprising:

(i) providing a Schizosaccharomyces pombe (Sz. pombe) yeast cell comprising:

(a) a G-Protein Coupled Receptor (GPCR)-regulated signalling pathway which is derepressed during the mitotic phase of cell growth;

(b) a reporter system for reporting a signal mediated by the GPCR-regulated signalling pathway;

(ii) producing a random peptide within the yeast cell; and

(iii) noting the output of the reporter system, for example by measuring an amount of reporter gene product produced by the yeast cell.

A still further aspect of the invention provides a compound capable of modulating GPCR-regulated activity identified by a method according to the invention. Assay kits comprising a yeast cell or isolated nucleic acid molecule as defined above are also provided.

M-cells do not normally express the P-factor mating pheromone (encoded by the map2 gene). P-factor is an unmodified peptide of 23 amino acids that is initially synthesised as a precursor containing an N-terminal signal sequence and four tandem copies of the mature pheromone. The signal sequence is lost after targeting the precursor into the secretory pathway and the precursor is then processed into the individual subunits before being released into the medium. A plasmid-based map2 construct that contains a single copy of the pheromone peptide and is expressed under the control of the nmt1 promoter has been prepared. Reporter strains containing the plasmid secrete P-factor when grown in thiamine-free medium and this elicits an autocrine response in the yeast cell in which the P-factor produced by the cell stimulates the pheromone receptor expressed in the same cell.

A further aspect of the invention therefore provides a yeast cell containing such a construct. Restriction sites may be provided within the construct to allow the P-factor sequence to be replaced by an alternative peptide sequence that is then secreted into the medium. Introducing random sequences into this construct produces a library of yeast strains in which each individual releases a different peptide, and allows random peptides to be assayed for their ability to act as autocrine inducers.

Strains of cells of, and useful in, the invention may be termed “reporter strains”.

Another feature of the invention is that it provides a method of determining whether a GPCR is coupled to the intracellular signalling machinery even in the absence of a ligand. Such a method is particularly useful for investigating orphan GPCRs, for which the natural ligand may not be known. The method is based on the as yet unexplained observation that the ligand-independent reporter system response is higher in a cell lacking a coupled receptor than it is in a comparable cell having a coupled receptor.

According to this aspect of the invention, there is therefore provided a method of determining whether a G-Protein Coupled Receptor (GPCR) is coupled to a cell signalling pathway, the method comprising comparing the ligand-independent reporter system output of a Schizosaccharomyces pombe (Sz. pombe) yeast cell comprising:

(i) a G-Protein Coupled Receptor (GPCR)-regulated signalling pathway which is derepressed during the mitotic phase of cell growth;

(ii) a reporter system for reporting a signal mediated by the GPCR-regulated signalling pathway;

with the reporter system output of a reference cell which lacks a functional GPCR.

The reference cell, which itself forms another aspect of the invention, will generally be a Schizosaccharomyces pombe (Sz. pombe) yeast cell comprising:

(i) a G-Protein Coupled Receptor (GPCR)-regulated signalling pathway which is derepressed during the mitotic phase of cell growth, wherein the GPCR is absent or otherwise rendered non-functional;

(ii) a reporter system for reporting a signal mediated by the GPCR-regulated signalling pathway.

The reporter system will be expected to give an output indicative of higher activity from the reference cell than from the cell under investigation if the GPCR in the cell under investigation is coupled to the signalling pathway.

Preferred features of each aspect of the invention are as for each other aspect, mutatis mutandis.

The invention will now be described by way of example only, with reference to the following figures:

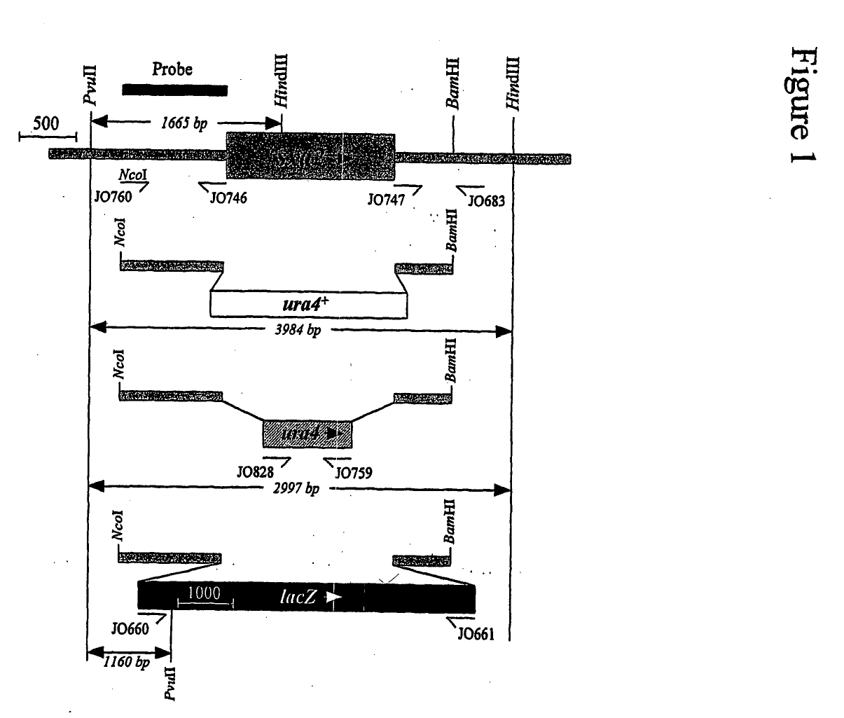

FIG. 1. Schematic diagram showing the identification and step-wise replacement of the sxa2 gene with ura4⁺, and the sxa2>ura4 and sxa2>lacZ reporter genes.

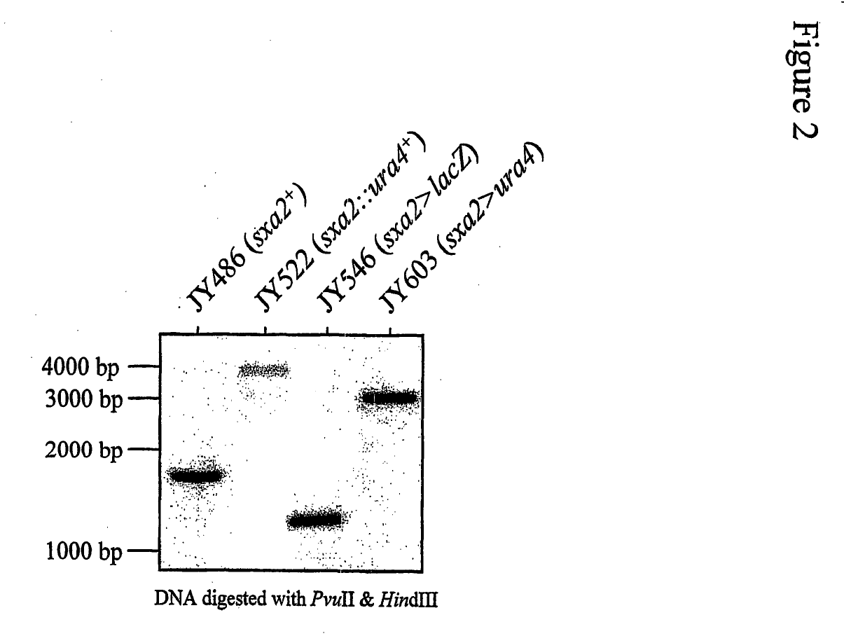

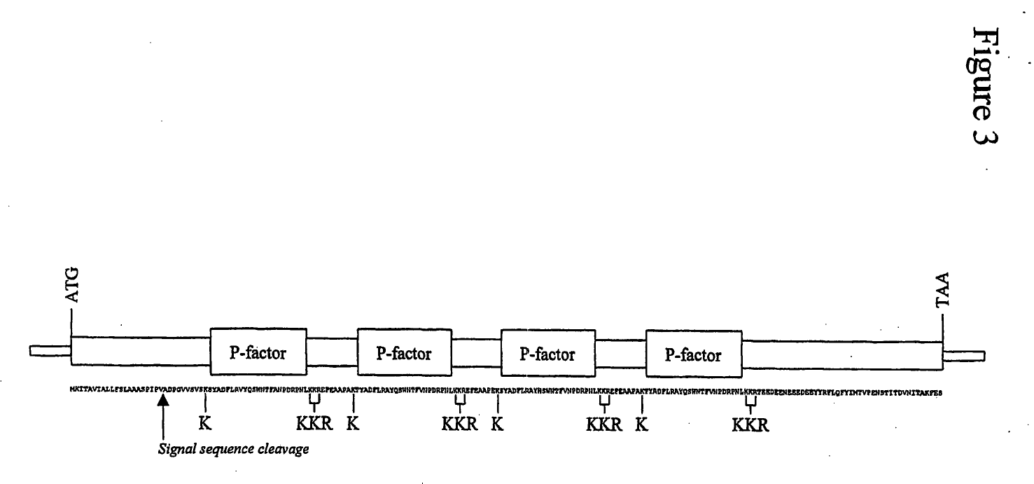

FIG. 2. Southern Blot of a PvuII and HindIII digest of the constructs shown schematically in FIG. 1.

FIG. 3. Schematic diagram of the arrangement of the map2 gene product

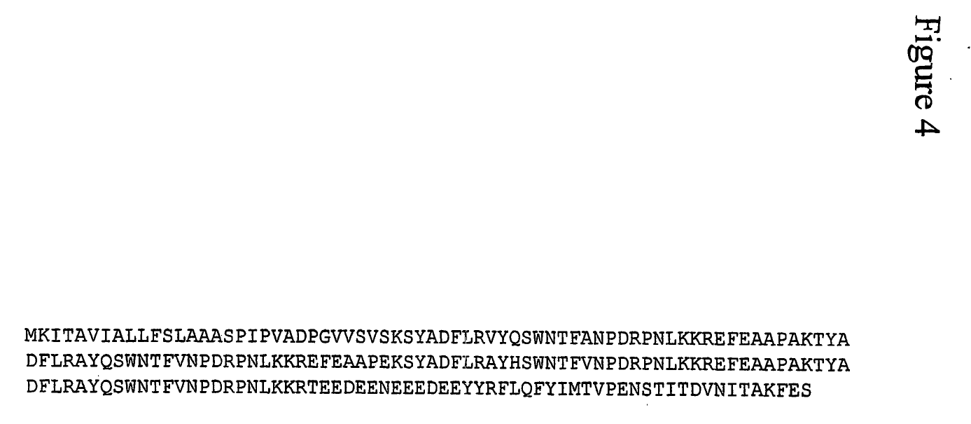

FIG. 4. Amino acid sequence of the map2 gene product.

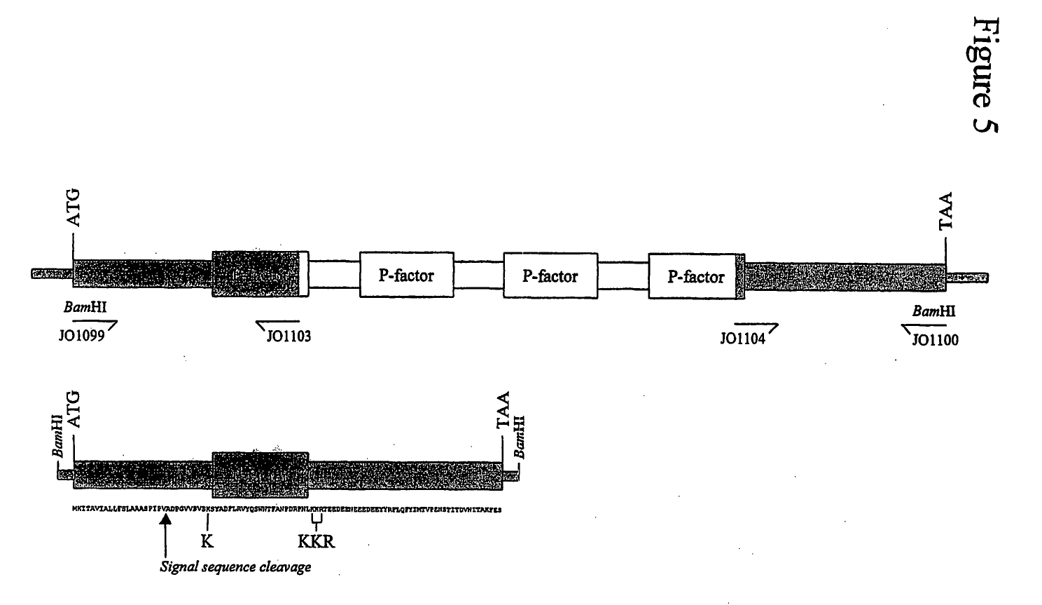

FIG. 5. Schematic diagram showing the construction of a construct containing only one copy of the P-factor gene (the mono P construct).

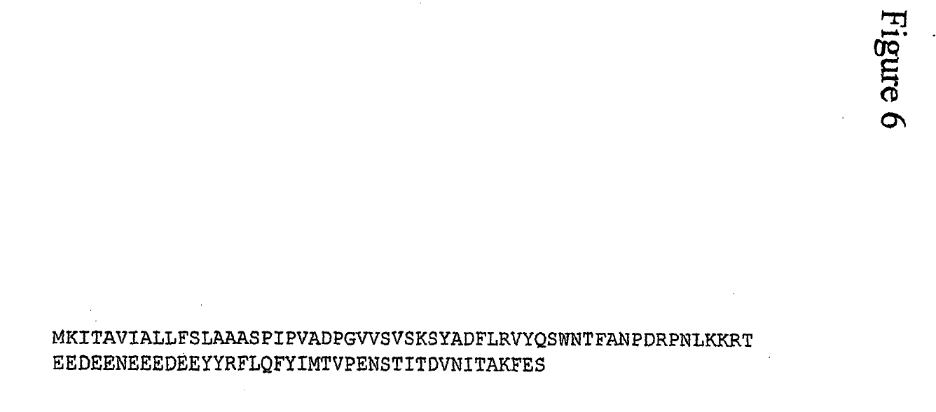

FIG. 6. Amino acid sequence of the mono P construct.

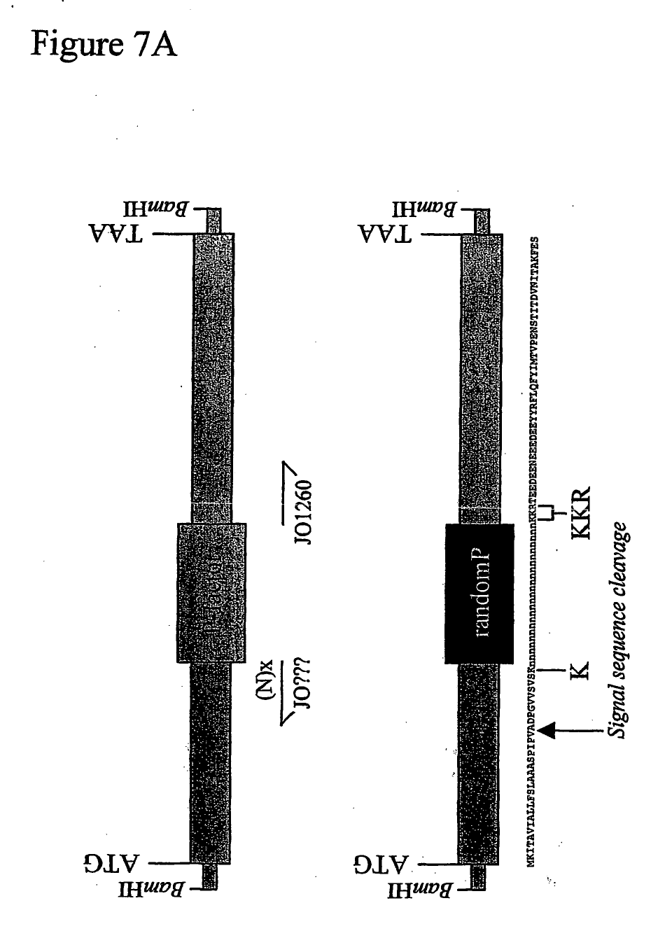

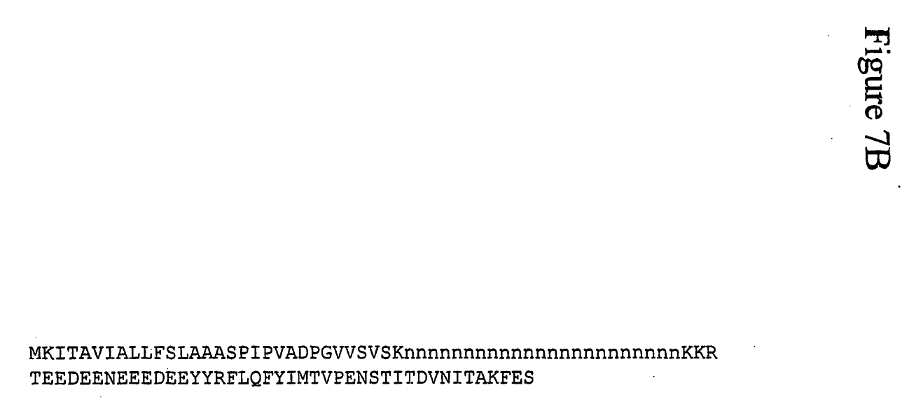

FIG. 7A. Schematic diagram showing the replacement of a P-factor gene with a nucleic acid sequence encoding a random peptide, Where “n” is an unknown amino acid.

FIG. 7B. Amino acid sequence of the modified P-factor gene product encoding a random peptide, where “n” is an unknown amino acid.

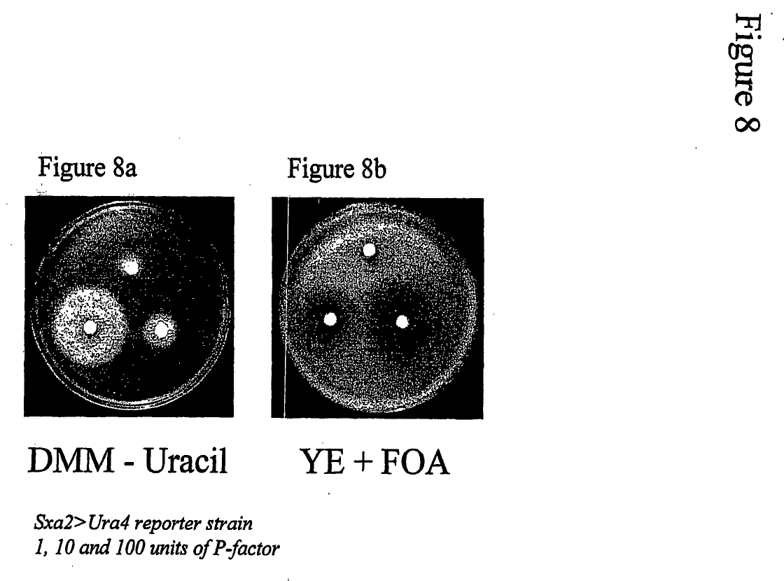

FIG. 8. Positive and negative selection using the ura4 reporter gene: a) Growth of yeast cells on plates without uracil upon stimulation with P-factor; b) Inhibition of growth on FOA plates: Yeast cells are stimulated with 1, 10 and 100 units of P-factor.

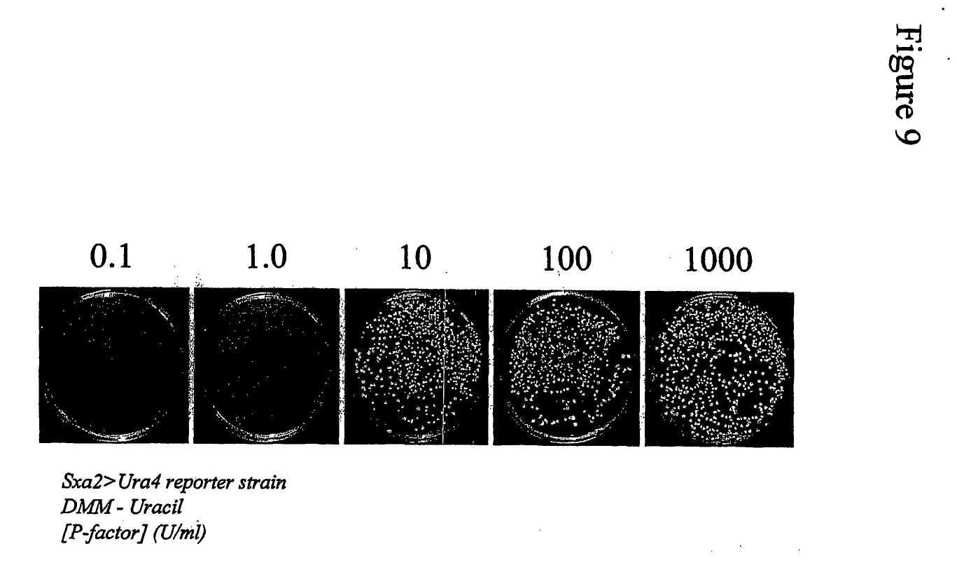

FIG. 9. Growth of sxa2>ura4. yeast cells on plates without uracil. The yeast cells are stimulated with between 0.1 and 1000 units/ml. P-factor.

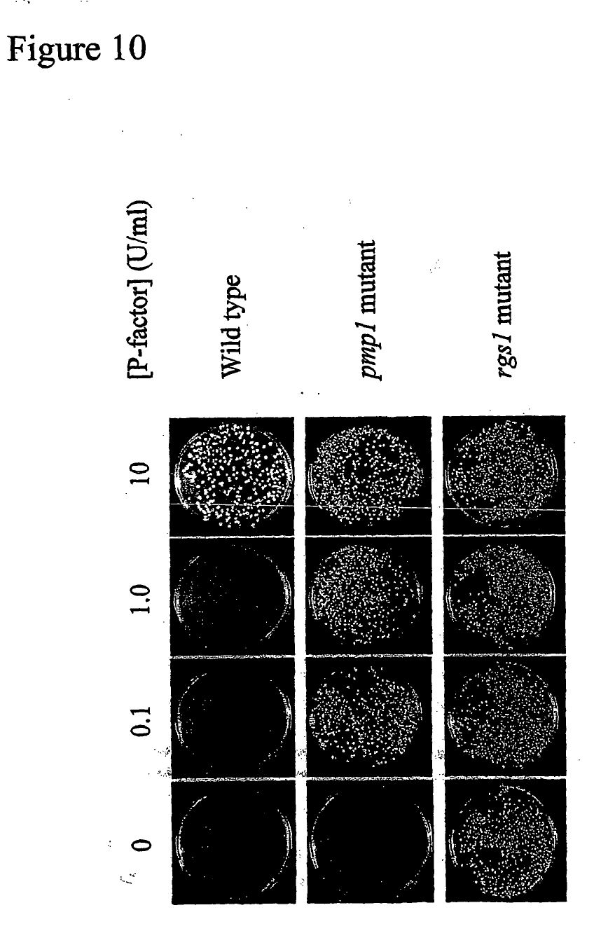

FIG. 10. Identification of mutants having enhanced sensitivity to P-factor stimulation. sxa2>ura4 cells were grown on plates lacking uracil.

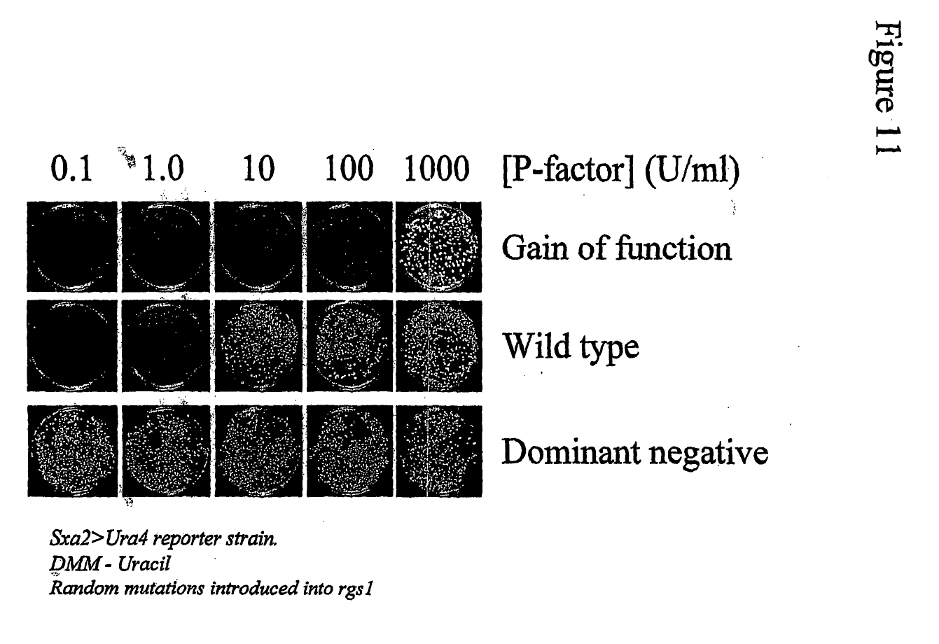

FIG. 11. Identification and characterisation of rgs1 mutants using the sxa2>ura4 strain.

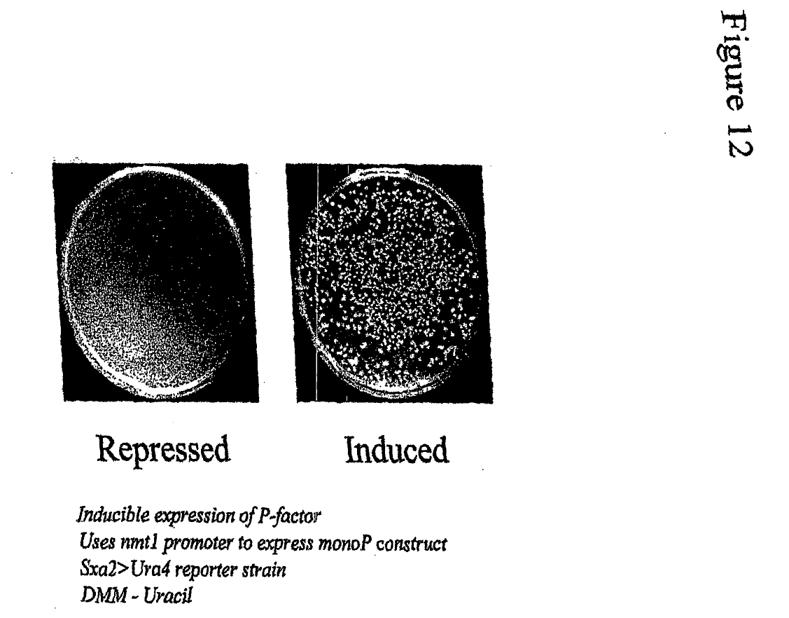

FIG. 12. Thiamine-inducible expression of P-factor using the sxa2>ura4 reporter strain and a thiamine-inducible P-factor construct.

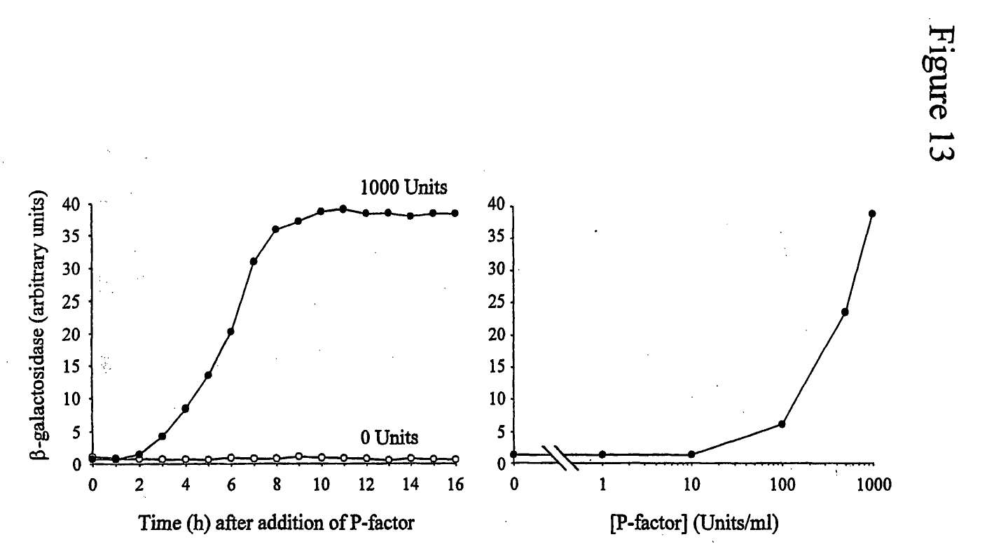

FIG. 13. P-factor stimulation of β-galactosidase in the sxa2>lacZ reporter strain

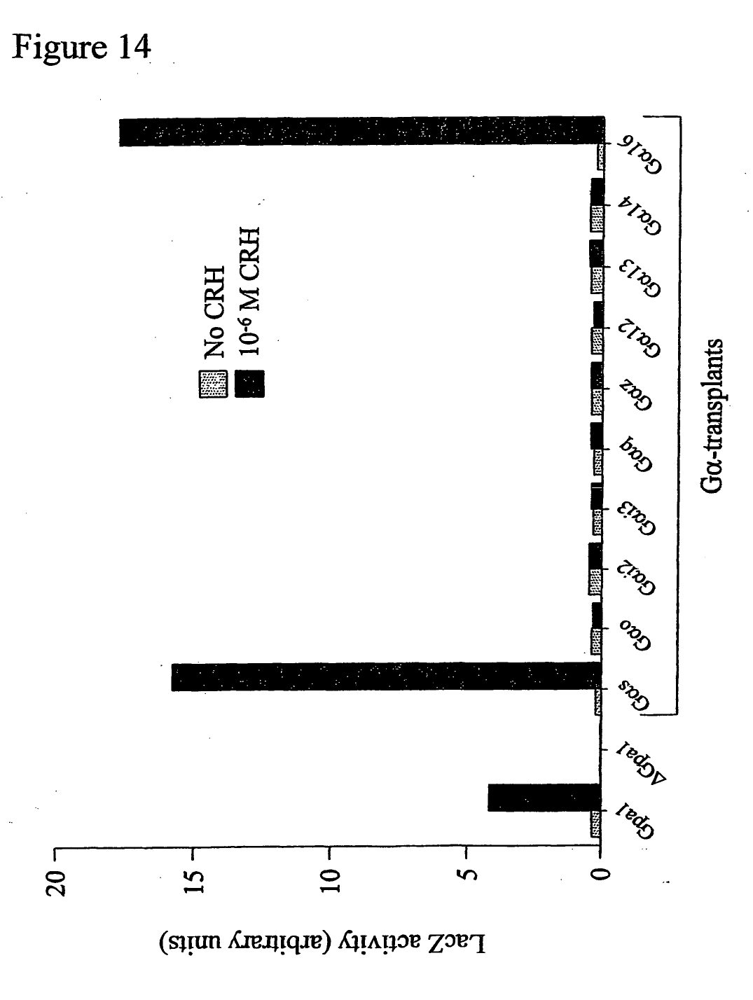

FIG. 14. Coupling of the human CRH receptor in Sz. pombe strains containing various Gα-transplants.

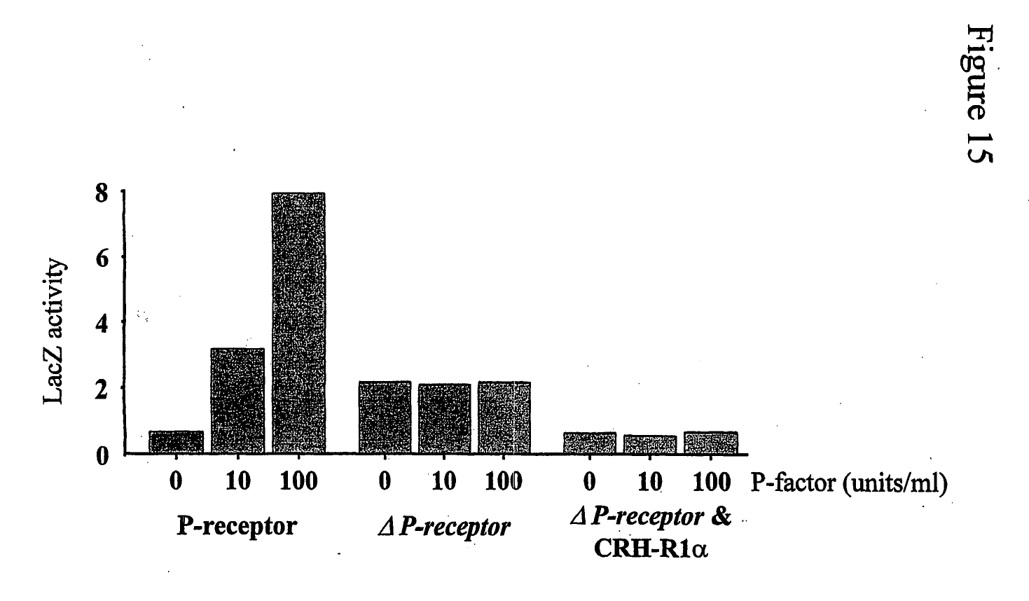

FIG. 15. Demonstrating the coupling of a receptor in the absence of its ligand.

### METHODS

All manipulations were by standard methods (see, for a general review, Davey et al., 1995). Reagents were obtained from common laboratory suppliers and used according to the manufacturer's recommendations. Unless stated otherwise, the polymerase chain reaction (PCR) was performed using Pwo DNA polymerase (from Pyrococcus woesei; supplied, by Boehringer Mannheim) as this has a 3′-5′ exonuclease (proof-reading) activity and reduced the introduction of errors during amplification. TAQ polymerase (from Thermus aquaticus; supplied by Boehringer Mannheim) was used for PCR with primers containing random sequences.

The yeast strains identified below are merely examples. Other suitable strains can be readily identified or produced using techniques known in the art.

Yeast Strains

JY271 is h⁻, cyrl::ura4, ade6-M216, leul-32, ura4-D18 and is equivalent to JZ300 (Maeda et al., 1990). This is an Mell but not stable and can switch mating type. The cyr1 gene (encoding adenylate cyclase) was disrupted by insertion of the ura4 gene (pDAC5), resulting in a cell which requires adenine and leucine for growth.

JY330 is mat1-P, Δmat2/3::LEU2⁻, leu1-32. The mat2-P and mat3-M donor mating cassettes were deleted by insertion of LEU2 (Klar and Miglio, 1986) and a LEU2⁻ isolate was then identified (Klar and Bonaduce, 1991).

JY444 is mat1-M, Δmat2/3::LEU2⁻, leu1-32, ura4-D18 and is a stable M-cell that requires leucine and uracil for growth.

The ura4 cassette used to disrupt the cyrl gene in JY271 was removed by standard techniques to create JY271B. JY271B is h⁻, cyrl-D51, ade6-M216, leul-32, ura4-D18.

This is an M-cell but not stable and can switch mating type. The cyr1 gene (adenylate cyclase) is disrupted. The cell requires adenine, leucine and uracil for growth.

JY271B was crossed with JY330 to generate JY361. JY361 is mat1-P, Δmat2/3::LEU2⁻, leu1-32, ade6-M216, ura4-D18, cyr1-D51. This is a stable P-cell which the cyr1 gene (adenylate cyclase) is disrupted. The cell requires adenine, leucine and uracil for growth.

JY361 was crossed with Y444 to generate JY486. JY486 is matl-M, Δmat2/3::LEU2⁻, leul-32, ade6-M216, ura4-D18, cyrl-D51. This is a stable M-cell in which the cyr1 gene (adenylate cyclase) is disrupted. The strain requires adenine, leucine and uracil for growth.

The sxa2 gene in JY486 was disrupted using a ura4⁺ cassette to generate JY522. The manipulation of the sxa2 gene is described in more detail below. The disruption cassette was a NcoI-to-BamHI fragment from JD883. JY522 is mat1-M, Δmat2/3::LEU2⁻, leu1-32, ade6-M216, ura4-D18, cyr1-D51, sxa2::ura4⁺. This is a stable M-cell in which the cyr1 gene (adenylate cyclase) is disrupted. The sxa2 gene (encodes a serine carboxypeptidase) is also disrupted. The strain requires adenine and leucine for growth.

The disrupted sxa2 gene in JY522 was replaced with the sxa2>lacZ reporter to generate JY546. The sxa2>lacZ reporter construct is from JD954. JY546 is matl-M, Δmat2/3::LEU2⁻, leul-32, ade6-M216, ura4-D18, cyrl-D51, sxa2>lacZ. This is a stable M-cell in which the cyrl gene (adenylate cyclase) is disrupted. The strain has an sxa2>lacZ reporter integrated at the sxa2 locus and expresses lacZ in response to pheromone stimulation. This strain requires adenine, leucine and uracil for growth.

The disrupted sxa2 gene in JY522 was also replaced with the sxa2>ura4 reporter to generate JY603. The sxa2>ura4 reporter construct is from JD929. JY603 is matl-M Δmat2/3::LEU2⁻, leul-32, ade6-M216, ura4-D18, cyrl-D51, sxa2>ura4, and is a stable M-cell. The cyr1 gene (adenylate cyclase) is disrupted. This has an sxa2>ura4 reporter integrated at the sxa2 locus and expresses ura4 in response to pheromone stimulation. The strain requires adenine and leucine for growth.

Constructing the sxa2>Reporter Strains

FIGS. 1 and 2 summarise the methods used to manipulate the sxa2 gene and promoter.

The sxa2 ORF was first replaced with a 1.8 kb Sz. pombe ura4+ cassette (Grimm et al., 1988). The complete sxa2 locus was amplified by PCR using the sense primer JO760 (ggggggtacCATGGCTAGAAATCCGCCATTGTGTG; lower-case letters are not complementary to sxa2 but the oligonucleotide includes a KpnI site [ggtac*C] and an NcoI site [c*CATGG] where digestion leaves ends that are fully homologous to the. chromosomal sequence) and the antisense primer JO683 (CTTCTCGTAAAGGCACATTGACGG, complementary to a region immediately downstream of the BamHI site at position 2043). The resulting PCR product was cloned into the KpnI and BamHI sites of pSP72 (Promega) to generate JD808 (pSP72 containing the sxa2 locus SEQ ID 31). This was used as template for PCR with JO746 (TGAAAAGAGAGACAATG; antisense primer complementary to a region immediately upstream of the ATG initiator codon for sxa2) and JO745 (TAAAAGTTTAATATC; sense primer complementary to a region that includes the TAA stop codon for sxa2) and the product ligated to the ura4⁺ cassette (to generate JD857, pSP72 containing a construct suitable for disruption of sxa2 SEQ ID 32) or to PCR products corresponding to either the lacZ ORF (to generate JD954, pSP72 containing the sxa2>lacZ reporter construct SEQ ID 33) or the ura4 ORF (to generate JD929, pSP72 containing the sxa2>ura4 reporter construct SEQ ID 34). The lacZ ORF was prepared by amplification using the sense primer JO660 (ATGCAGCTGGCACGACAGGTTTCCCGAC; includes the ATG initiator codon and next 25 bases of the lacZ ORF) and the antisense primer JO661 (TTTTTGACACCAGACCAACTGGTAATGGTAGC; complementary to the 3′ end of the lacZ ORF but lacks the stop anticodon). The ura4 ORF was prepared by amplification using the sense primer JO828 (ATGGATGCTAGAGTATTTC; includes the ATG initiator codon and next 16 bases of the ura4 ORF) and the antisense primer JO759 (ATGCTGAGAAAGTCTTTGC; complementary to the 3′ end of the ura4 ORF but lacks the stop anticodon).

JY486 (a mating stable M-cell lacking cyrl) was transformed with a NcoI-BamHI fragment corresponding to the sxa2::ura4⁺ construct (isolated from JD857), and stable Ura4⁺ transformants were initially screened by PCR and replacement of the sxa2 locus was confirmed by Southern blot (FIG. 2). A correct sxa2::ura4⁺ disruptant (JY522) was then transformed with the NcoI-BamHI fragments corresponding to the sxa2>lacZ reporter (isolated from JD954) or the sxa2>ura4 reporter (isolated from JD929). Stable Ura⁻ transformants were selected by their ability to grow in the presence of 5′fluoro-orotic acid (Boeke et al., 1987) and homologous integration of the reporter constructs at the sxa2 locus was confirmed by Southern blot for JY546 (sxa2>lacZ) and JY603 (sxa2>ura4). Southern blot analysis was performed on genornic DNA digested with PvuII & HindIII and a probe corresponding to the 5′ untranslated region of sxa2.

Constructing the Gα-Transplants

This was undertaken using techniques well known in the art. A SpeI-PstI fragment from gpa1 (SEQ ID 1, 15) was cloned into the SpeI and PstI sites of the plasmid pKS-Bluescript (Stratagene). This 324 bp fragment contains the last 24 residues of Gpa1 and 250 base pairs from the 3′ untranslated region of the gpa1 gene. The resulting clone (JD1647) was then used as template for a series of polymerase chain reactions using oligonucleotide primers that made the desired changes to the residues at the C-terminus of Gpa1. Each reaction used the antisense primer JO1354 (TAGATTGTTGGACATAATCGTATCTTGAACGG; complementary to a region from position 1206 to position 1175 relative to the intiator ATG of gpa1) and an appropriate sense primer that introduced the desired changes and was complementary to the region immediately downstream of the Gpa1 open reading frame; JO1344 for the Gαq-transplant (gaatataatcttgttTAGATGAATTMTCCTTAAC, lower case letters, change the last 5 residues of Sz. pombe Gpa1 to EYNLV), JO1345 for the Gαs-transplant (caatatgaacttcttTAGATGAATTTTTCCTTAAC; change last 5 residues of Gpa1 to QYELL), JO1346 for the Gαo-transplant (ggatgcggactttatTAGATGAATTTTTCCTTAAC, change last 5 residues of Gpa1 to GCGLY), JO1347 for the Gαi2-transplant (gattgcggactttttTAGATGAATTTlTCCTTAAC, change last 5 residues of Gpa1 to DCGLF), JO1348 for the Gαi3-transplant (gaatgcggactttatTAGATGAATTTTTTCCAAC, change last 5 residues of Gpa1 to ECGLY), JO1349 for the Gαz-transplant (tatattggactttgcTAGATGAATTTTTCCTTAAC, change last 5 residues of Gpa1 to YIGLC), JO1350 for the Gα12-transplant (gatattatgcttcaaTAGATGAATTTTTCCTTAAC, change last 5 residues of Gpa1 to DIMLQ), JO01351 for the Gα13-transplant (caacttatgcttcaaTAGATGAATTTTTCCTTAAC, change last 5 residues of Gpa1 to QLMLQ), JO1352 for the Gα14-transplant (gaatttaatcttgttTAGATGAATTTTCCTTAAC, lower case letters change the last 5 residues of Gpa1 to EFNLV) and JO1353 for the Gα16-transplant (gaaattaatcttcttTAGATGAATTTTTCCTTAAC, change last 5 residues Gpa1 to EINLL).

The PCR products were sequenced to confirm that the correct changes had been made and were then used to replace the equivalent SpeI-PstI fragment from JD1645 (pSP71-Gpa1). JD1645 contains the complete gpa1 sequence from an EcoRI site at position −676 (relative to the initiator ATG) to a BglII site at position 1938. This generated a series of plasmids containing the modified gpa1 sequences; JD1649 (Gαq-transplant SEQ ID 17, 03), JD1650 (Gαs-transplant SEQ ID 16, 02), JD1651 (Gαo-transplant SEQ ID 18, 04), JD1652 (Gαi2-transplant SEQ ID 19, 05), JD1653 (Gαi3-transplant SEQ ID 20, 06), JD1654 (Gαz-transplant SEQ ID 21, 07), JD1655 (Gα12-transplant SEQ ID 22, 08), JD1656 (Gα13-transplant SEQ ID 23, 09), JD1657 (Gα14-transplant SEQ ID 24, 10) and JD1658 (Gα16-transplant SEQ ID 25, 11). The coding regions for the different Gα-transplants were isolated as EcoRI-BglII fragments and used separately to transform the yeast strain JY1170. JY1170 is matl-M, Δmat2/3::LEU2⁻, leul-32, ade6-M216, ura4-D18, cyrl-D51, mam2-D10, gpa1::ura4⁺, sxa2>lacZ. This is a derivative of the standard JY546 reporter strain but it lacks the mam2 gene (encodes the P-factor receptor) and the gpa1 gene has been disrupted by insertion of a ura4⁺ cassette. Ura⁻ transformants were selected on fluoro-orotic acid and Southern blot analyses were used to confirm integration of the Gα-transplant constructs at the gpa1 locus. This generated a series of Sz. pombe sxa2>lacZ reporter strains lacking the mam2 pheromone receptor but containing integrated Gα-transplants; JY1165 (Gαq-transplant), JY1157 (Gαs-transplant), JY1158 (Gαo-transplant), JY1159 (Gαi2-transplant), JY1160 (Gαi3-transplant), JY1161 (Gαz-transplant), JY1162 (Gα12-transplant), JY1163 (Gα13-transplant), JY1164 (Gα14-transplant) and JY1167 (Gα16-transplant).

Generating Peptides for Autocrine Signalling

M-cells do not normally express the P-factor mating pheromone (encoded by the map2 gene). P-factor is an unmodified peptide of 23 amino acids that is initially synthesised as a precursor containing an N-terminal signal sequence and four tandem copies of the mature pheromone. The signal sequence is lost after targeting the precursor into the secretory pathway and the precursor is then processed into the individual subunits before being released into the medium. A plasmid-based map2 construct that contains a single copy of the pheromone peptide and is expressed under the control of the thiamine-regulated nmtl promoter shown schematically in FIGS. 3 to 6 was prepared.

This was undertaken using techniques well known in the art. Reporter strains containing the plasmid secrete P-factor when grown in thiamine-free medium (the nmtl promoter is on) and this elicits an autocrine response in the strain. Restriction sites within the construct allow the P-factor sequence to be replaced by an alternative peptide sequence that would then be secreted into the medium (FIGS. 7A and 7B). Introducing random sequences into this construct produces a library of strains in which each individual releases a different peptide. This allows ligands capable of binding to the pheromone receptor or another GPCR to be identified.

Expression and Application of Reporter Gene Constructs

Demonstration of a sxa2>ura4 Reporter Construct

JY603 yeast cells containing the construct were spread as a confluent layer of cells (about 10⁷ cells on each plate) on DMM medium lacking uracil. Paper disks were placed on the dried surface of the cells and aliquots containing different amounts of P-factor were added to each disk. The plates were then incubated at 29° C. for 3 days. FIG. 8A shows that cells are not normally able to grow in the absence of uracil but the P-factor induces expression of the sxa2>ura4 reporter and allows a growth of cells around the disks. The halo is largest around the disk containing 100 units of P-factor.

This is also demonstrated in FIG. 4, except that a series of plates containing different concentrations of P-factor were used. AU of the plates received the same number of yeast cells (about 2,000 cells per plate). The cells are not normally able to grow in the absence of uracil but the P-factor induces expression of the sxa2>ura4 reporter and allows cells to form colonies. There are no colonies on the plates containing 0.1 or 1.0 units/ml. but colonies form on plates containing P-factor with at least 10 units/ml.

FIG. 8B shows plates which contain uracil and 5-fluoro-orotic acid (FOA). Cells not expressing ura4 are able to grow on these plates but those expressing ura4 convert the FOA into a toxic compound and die. There are clear halos of no growth around the disk containing the P-factor. The halo is largest around the disk containing 100 units of P-factor.

Identification of Mutants

The sxa2>ura4 reporter system allows the identification of mutants. Cells can be randomly mutagenised and spread on plates containing P-factor at 0.1 units/ml to identify mutations that make the cells more sensitive to stimulation. FIG. 10 shows two of these mutations. This approach can also be used to identify mutant forms of various proteins involved in regulating the signalling pathway as shown in FIG. 11.

The sxa2>ura4 reporting strain was randomly mutagenised and then spread on plates lacking uracil but containing P-factor at 0.1 units/ml. The wild-type cells do not normally grow on these plates, since they require P-factor at a concentration of at least 10 units/ml. A number of mutants that had increased sensitivity to signalling and were now able to grow at a low level of P-factor were identified. Two of these mutants have been characterised as being rgs1 and pmp1. The pmp1 mutant does not grow on plates lacking P-factor but grows on a very low level of P-factor. This demonstrates that it is a hypersensitive mutant. In contrast, the rgs1 mutant grows even in the absence of P-factor, showing that it is a constitutive responder (that is, it expresses the reporter gene in the absence of stimulation by ligand).

The Applicants mutated the cloned rgs1 gene to isolate mutant forms of the protein with altered properties and screened for isolates that were either gain of function mutants (have increased activity relative to the normal Rgs1 protein) or dominant negative mutants (inactive mutants that inhibit the activity of the normal Rgs1 protein in the same cell).

Autocrine Signalling

The inventors modified a version of the map2 gene that encodes the P-factor precursor was modified so that it contained a single copy of the P-factor (“mono P”). This is cloned into a plasmid so that expression of the P-factor was under the control of the thiamine-repressible nmtl promoter. The plasmid was introduced into M-cells which do not normally produce P-factor but are able to respond to P-factor. The cells were spread on plates lacking uracil but containing either no thiamine or 5 μM thiamine (see FIG. 12). The thiamine induces expression and release of the P-factor, causing autocrine signalling of the cell. Thins results in the expression of the sxa2>ura4 reporter.

The lacZ Reporter

The sxa2>lacZ reporter strain was grown in the presence of varying amounts of P-factor. The amount of β-galactosidase released was assayed using o-nitrophenyl-β-D-galactopyranoside and measuring the amount of product at OD₄₂₀. FIG. 13 shows the effect of adding P-factor over time and with increasing concentration The concentration-dependent assay was measured 16 hours after adding the pheromone.

Coupling of a Human GPCR

By way of example, the human receptor for corticotrophin releasing hormone (CRH, also known as corticotrophin releasing factor or CRF) was expressed in the Sz. pombe sxa2>lacZ reporter strains containing either Gpa1 or the various Gα-transplants. The yeast strains were transformed with pREP3X:CRH-R1 (SEQ ID 30), a plasmid that places the CRH receptor (see SEQ ID 28 and SEQ ID 14) type 1α under the control of the nmt1 promoter. Transformants were grown in the absence of thiamine (to allow expression of the receptor) and then exposed to CRH at 10⁻⁶ M (control cells were exposed to solvent lacking CRH). The amount of β-galactosidase released was assayed after 16 hours using o-nitrophenyl-β-D-galactopyranoside (FIG. 14). A low level of coupling was observed with the endogenous Gpa1 but this was considerably improved in the Gαs- and Gα16-transplants.

CRH is a 41-residue peptide that is a major regulator of the body’s stress axis. Although it has several functions, its best characterised role is in initiating pituitary-adrenal responses to stress, an effect mediated through CRH-R1α (Vale et al., 1981). This receptor normally functions through Gαs, resulting in activation of adenylate 2cyclase and increased levels of cAMP (Giguere et al., 1982; Bilezikjan and Vale, 1983; Grammatopoulos et al., 1996). The observed coupling to the Gαs-transplant is consistent with the activity of the CRH-R1α receptor in mammalian cells. Gα16 is known to interact with a wide range of GPCRs (Milligan et al., 1996).

There are no reports of the coupling of the human CRH receptor to the signalling machinery in the budding yeast S. cerevisiae and a direct comparison with the Sz. pombe reporter strains reported here is therefore not possible. It is perhaps significant however that a peptide ligand similar to CRH appears unable to gain access to receptors at the surface of S. cerevisiae cells (Baranski et al., 1999). The C5a chemoattractant receptor is functional in S. cerevisiae but can only be stimulated by its ligand (a 74-residue peptide) when both the receptor and the ligand are expressed in the same cell. Such autocrine stimulation is required because the C5a ligand is unable to traverse the S. cerevisiae cell wall.

Sz. pombe is also surrounded by a cell wall but it has a very different structure to that surrounding S. cerevisiae (for reviews, see, Osurmi, 1998; Smits et al., 1999) and previous studies of intoxication by diphtheria toxin demonstrated that the two have quite different permeability properties. Diphtheria toxin, secreted by certain strains of Corynebacterium diphtheriae, catalyses the ADP-ribosylation of eukaryotic aminoacyl transferase II (EF-2) using NAD as substrate. This reaction forms the basis for its toxicity toward eukaryotic organisms. Intoxication requires the entry of the toxin into the cytoplasm after internalisation by endocytosis. Studies have investigated the effects of diphtheria toxin on protein synthesis in S. cerevisiae (Murakami et al., 1982) and Sz. pombe (Davey, 1991). Although the Sz. pombe cells were sensitive to the toxin, intact S. cerevisiae cells were resistant to its effects. In contrast, S. cerevisiae spheroplasts (cells in which the cell wall has been enzymatically removed) were sensitive to the toxin, suggesting that the failure of the toxin to enter intact cells was due to its inability to cross the cell wall. Diphtheria toxin is a heterodimer composed of an N-terminal A fragment (molecular weight 24,000 daltons) that is enzymatically active and a C-terminal B fragment (molecular weight 39,000 daltons) that has no apparent enzymatic activity but is required for toxicity.

The apparent greater permeability of the cell wall in Sz. pombe night be invaluable for the study of receptors with large or complex ligands and could provide an advantage over the use of S. cerevisiae in such situations.

Demonstrating Coupling of a Receptor in the Absence of its Ligand

The lack of available ligands for orphan GPCRs makes it difficult to confirm that such receptors are coupled to the signalling machinery. High throughput screens are therefore performed without the confidence that a ligand which would normally stimulate the receptor would be identified as being active. The inventors have observed an interesting feature of the Sz. pombe reporter strains that can indicate whether a GPCR is coupled to the intracellular signalling machinery even in the absence of its ligand. Such knowledge generates confidence prior to performing high throughput screens. An example of this feature is shown in FIG. 15. When a S. pombe sxa2>lacZ reporter strain expressing the normal P-factor pheromone receptor is exposed to P-factor, there is a ligand-dependent induction of β-galactosidase. As expected, a similar strain lacking the P-factor receptor fails to exhibit ligand-dependent induction of the sxa2>lacZ reporter. However, the ligand-independent expression of the sxa2>lacZ reporter (i.e. the level of β-galactosidase activity observed in the absence of P-factor) is considerably higher in the strain lacking the P-factor receptor than in the strain containing the P-factor receptor. Expressing the human CRH-R1α receptor in the strain lacking the P-factor receptor reduces the ligand-independent expression of the sxa2>lacZ reporter back to the levels observed in the strain containing the P-factor receptor. As there is no P-factor receptor in this strain, addition of P-factor fails to induce further expression of the sxa2>lacZ reporter.

This observation is not limited either to the particular reporter system or to the CRH-R1α receptor and has been observed with many other GPCRs that were then subsequently shown to be coupled to the Sz. pombe signalling machinery. A molecular explanation for this effect is not available but it could simply reflect the ability of the receptor to sequester the heterotrimeric G proteins and prevent inappropriate activation of the downstream effector protein(s). Whatever the explanation, the ability of a receptor to reduce the ligand-independent expression of β-galactosidase appears to reflect its ability to couple to the Sz. pombe signalling machinery.

Applications

Identify Agonists and Antagonists for GPCRs, Including Orphan GPCRs

Reporter strains expressing either a characterised or an orphan receptor can be used in a variety of assays to identify ligands that affect signalling through the receptors. Agonists will elicit a response in the strain while antagonists could be identified by their ability to inhibit stimulation by a ligand known to activate the receptor. Both peptides and small molecules can be screened and assays might be either liquid- or plate-based, depending on the reporter gene used. Screens for peptide ligands could exploit the autocrine signalling of strains producing a library of random peptides.

Identify Intracellular Signal Regulators and Modified Regulators with Altered Activities

Regulators of the intracellular response pathway can be identified by their ability to influence signalling in the reporter strains. Over-expression of these proteins will either reduce or increase signalling depending on whether they are positive or negative regulators. A number of mammalian regulators are known to be active in yeast. Regulators identified through these screens can then be mutagenised and the reporter strains used to identify isolates with altered activities. Gain-of-function mutants, for example, would have increased abilities to regulate signalling while dominant-negative mutants would not only be inactive but would also inhibit the activity of the wild type regulator. These mutants could then be introduced back into mammalian systems to assess their ability to regulate other signalling pathways.

Identify Reagents that Modulate Signalling

Random peptides can be expressed in the cytoplasm of the reporter strains and assayed for their ability to regulate signalling. These ‘peptamers’ could interact directly with components from the signalling pathway or might exert their effect through the intracellular signal regulators mentioned earlier. The screen is not limited to peptide regulators and would also identify small molecules that could influence signalling.

Schizosaccharomyces pombe strain JY546 was deposited under the Budapest Treaty at the National Collection of Yeast Cultures, Norwich,. United Kingdom on 27 Oct. 2000. It has been given Accession Number NCYC 2984.

References

Allshire, R. C., Cranston, G., Gosden, J. R., Maule, J. C., Hastie, N. D. and Fantes, P. A. (1987) A fission yeast chromosome can replicate autonomously in mouse cells. Cell 50, 391-403.

Aono, T., Yanai, H., Mild, F., Davey, J. and Shimoda, C. (1994) Mating Pheromone-induced Expression of the mat1-Pm gene of Schizosaccharomyces pombe: Identification of Signalling Components and Characterisation of Upstream Controlling Elements. Yeast 10, 757-770.

Arkinstall, S., Edgerton, M., Payton, M. and Maundrell, K. (1995) Co-expression of the neurokinin NK2 receptor and G-protein components in the fission yeast Schizosaccharomyces pombe. FEBS Letts. 375, 183-187.

Baldwin, J. M. (1994) Structure and function of receptors coupled to G proteins. Current Opinion Cell Biol. 6, 180-190.

Baranski, T. J., Herzmark, P., Lichtarge, O., Gerber, B. O., Trueheart, J., meng, E. V., Iiri. T., Sheikh, S. P. and Bourne, H. R. (1999) C5a receptor activation. Genetic identification of critical residues in four transmembrane helices. J. Biol. Chem. 274, 15757-15765.

Bass, R. T., Buckwalter, B. L., Patel, B. P., Pausch, M. N., Price, L. A., Strnad, J. and Hadcock, J. R. (1996) Identification and characterisation of novel somatostatin antagonists. Mol. Pharmacol. 50, 709-715.

Bilezikjian, L. M. and Vale, W. W. (1983) Glucocorticoids inhibit corticotropin-releasing factor-induced production of adenosine 3′,5′-monophosphate in cultured anterior pituitary cells. Endocrinology 113, 657-662.

Boeke, J. D., Trueheart, J., Natsoulis, G. and Fink, G. R. (1987) 5′-fluroorotic acid as a selective agent in yeast molecular genetics. Methods Enzymol. 152, 164-175.

Brown, A. J., Dyos, S. L., Whiteway, M. S., White, J. H. M., Watson, M -A. E. A., Marzioch, M., Clare, J. J., Cousens, D. J., Paddon, C., Plumpton, C., Romanos, M. A. and Dowell, S. J. (2000) Functional coupling of mammalian receptors to the yeast mating pathway using novel yeast/maiimalian G protein α-subunit chimeras. Yeast 16, 11-22.

Chambers, J. K., Macdonald, L. E., Sarau, H. M., Ames, R. S., Freeman, K., Foley, J. J., Zhu, Y., McLaughlin, M. M., Murdock, P., McMillan, L., Trill, J., Swift, A., Aiyar, N., Taylor, P., Vawter, L., Naheed, S., Szekeres, P., Hervieu, G., Scott, C., Watson, J. M., Murphy, A. J., Duzic, E., Klein, C., Bergsma, D. J., Wilson, S. and Livi, G. P. (2000) A G protein-coupled receptor for UDP-glucose J. Biol. Chem. 275, 10767-10771.

Conklin, B. R., Farfel, Z., Lustig, K. D., Julius, D. and Bourne, H. R. (1993) Substitution of three amino acids switches receptor specificity of Gαq to Gαi. Nature 363, 274-276.

Davey, J. (1991) Isolation and quanitation of M-factor, a diffusible mating factor from the fission yeast Schizosaccharomyces pombe. Yeast 7, 357-366.

Davey, J. and Nielsen 0. (1994) Mutations in cyr1 and pat1 reveal pheromone-induced G1 arrest in the fission yeast Schizosaccharomyces pombe. Curr. Genet. 26, 105-112.

Davey, J., Egel, R. and Nielsen, O. (1995) Pheromone procedures in fission yeast in Microbial Gene Techniques; Methods in Molecular Genetics 6B (Adolph, K. W., ed.) Academic Press, San Diego. pp 247-263.

Davey, L (1998) Fusion of a fission yeast. Yeast 14, 1529-1566.

Erickson, J. R., Wu, J. J., Goddard, G., Tigyi, G., Kawanishi, K, Tomei L. D. and Kiefer, M. C. (1998) Edg-21Vzg-1 couples to the yeast pheromone response pathway selectively in response to lysophosphatidic acid. J. Biol. Chem. 273, 1506-1510.

Erlenbach, I., Kostenis, E., Schmidt, C., Hamdan, F. F., Pausch, M. H. and Wess, J. (2001) Functional expression of M1, M3 and M5 muscarinic acetylcholine receptors in yeast J. Neurochem 77,1327-1337.

Ficca, A. G., Testa, L., and Valentini, G. P. T. (1995) The human β2-adrenergic receptor expressed in Schizosaccharomyces pombe retains its pharmacological properties. FEBS Letts. 377,140-144.

Giguere, V., Labrie, F., Cote, J., Coy, D. H., Sueiras-Diaz, J. and Schally, A. V. (1982) Stimulation of cyclic AMP accumulation and corticotropin release by synthetic ovine corticotropin-releasing factor in rat anterior pituitary cells: site of glucocorticoid action. Proc. Natl. Acad. Sci. USA 79, 3466-3469.

Grammatopoulos, D., Stirrat, G. M., Williams, S. A and Hillhouse, E. W. (1986) The biological activity of the corticotropin-releasing hormone receptor-adenylate cyclase complex in human myometrium is reduced at the end of pregnancy. J. Clin. Endocrinol. Metab. 81, 745-51.

Grimm, C., Kohli, J., Murray, J. and Maundrell, K. (1988) Genetic engineering of Schizosaccharomyces pombe: a system for gene disruption and replacement using the ura4 gene as a selectable marker. Mol. Gen. Genet. 215, 81-86.

Hamm, H. E., Deretic, D., Arendt, A, Have, P. A., Koenig, B. and Hofmann, K. P. (1988) Site of G protein binding to rhodopsin mapped with synthetic peptides from the α-subunit Science 241, 832-835.

Hildebrandt, V., Fendler, K., Heberle, J., Hofmann, A., Bamberg, E. and Büldt, G. (1993) Bacteriorhodopsin expressed in Schizosaccharomyces pombe pumps protons through the plasma membrane. Proc. Natl. Acad. Sci. USA 90, 3578-3582.

Hisch, J. P., Dietzel, C. and Kurjan, 1 (1991) The carboxyl terminus of Scg1, the Gα subunit involved in yeast mating, is implicated in interactions with the pheromone receptors. Genes Dev. 5, 467-474.

Jones, R. H., Moreno, S., Nurse, P. and Jones, N. J. (1988) Expression of the SV40 promoter in fission yeast: Identification and characterisation of an AP-1-like factor. Cell 53, 659-667.

Kajkowski, E. M., Price, L. A., Pausch, M, H., Young, K. H. and Ozenberger, B. A. (1997) Investigation of growth hormone releasing hormone receptor structure and activity using yeast expression technologies. J. Receptor Signal Transduction Res. 17, 293-303.

King, K, Dohlman, H. G., Thorner, J., Caron, M. G. and Lefkowitz, R. J. (1990) Control of yeast mating signal transduction by a mammalian β2-adrenergic receptor and Gs α-subunit. Science 250, 121-123.

Klar, A. J. S. and Bonaduce, M. J. (1991) swi6, a gene required for mating-type switching, prohibits meiotic recombination in the mat2-mat3 “cold-spot” of fission yeast Genetics 129, 1033-1042.

Klar, A. J. S. and Miglio, L. M. (1986) Initiation of meiotic recombination by double-strand DNA breaks in Schizosaccharomyces pombe. Cell 46, 725-731.

Klein, C., Paul. J. I., Sauve, K, Schmidt, M. M., Arcangeli, L., Ransom, J., Trueheart, J,. Manfredi, J. P., Broach, J. R. and Murphy, A. J. (1998) Identification of surrogate agonists for the human FPRL-1 receptor by autocrire selection in yeast. Nature Biotech. 16, 1334-1337.

Komatsuzki, K, Murayama, Y., Giambarella, U., Ogata, E, Seino, S. and Nishimoto, I. (1997) A novel system that reports the G-proteins linked to a given receptor: a study of the type 3 somatostatin receptor. FEBS Letts 406, 165-170.

Ladds, G., Rasmussen, E. M., Young, T., Nielsen, O. and Davey, J. (1996) The sxa2-dependent inactivation of the P-factor mating pheromone in the fission yeast Schizosaccharomyces pombe. Mol. Microbiol. 20,3542.

Liu, J., Coklin, B. R., Blin, N., Yun, J. and Wess, J. (1995) Identification of a receptor/G-protein contact site critical for signaling specificity and G-protein activation. Proc. Natl. Acad Sci. USA 92, 11642-11646.

Maeda, T., Mochizuki, N. and Yamamoto, M. (1990) Adenylyl cyclase is dispensable for vegetative cell growth in the fission yeast Schizosaccharomyces pombe. Proc. Natl. Acad Sci. USA 87,7814-7818.

Milligan, G., Marshal, F. and Rees, S. (1996) Gα16 as a universal G protein adapter: implications for agonist screening strategies. Trends Pharmacol. Sci 17,235-237.

Murakami, S., Bodley, J. W. and Livingston, D. M. (1982) Saccharomyces cerevisiae spheroplasts are sensitive to the action of diphtheria toxin Mol. Cell. Biol. 2, 588-592.

Olesnicky, N. S., Brown, A. J., Dowell, S. J. and Casselton, L. A. (1999) A constitutively active G-protein-coupled receptor causes mating self-incompatibility in the mushroom Coprinus. EMBO J. 18, 2756-2763.

Osumi, M. (1998) The ultrastncture of yeast: cell wall structure and formation. Micron 29, 207-233.

Palm, D., Munch, G., Malek, D., Dees, C. and Heckman, M. (1990) Identification of a Gs protein coupling domain to the beta-adrenoreceptor using site-specific synthetic peptides. Carboxyl terminus of Gαs is involved in coupling to the β-adrenoreceptors. FEBS Letts 261, 294-298.

Price, L. A., Kajkowski, E. M., Hadcock, J. R, Ozenberger, B. A. and Pausch, M. H. (1995) Functional coupling of a mammalian somatostatin receptor to the yeast pheromone response pathway. Mol. Cell. Biol. 15, 6188-6195.

Price, L. A., Strnad, J., Pausch, M. H. and Hadcock, J. R (1996) Pharmacological characterisation of the rat A_{2A} adenosine receptor functionally coupled to the yeast pheromone response pathway. Mol. Pharmacol. 50, 829-837.

Rasenick, M. M., Watanabe, M., Laarevic, M. B., Hatta, S. and Hamm, H. E. (1994) Synthetic peptides as probes for G protein function. J. Biol. Chem. 269, 21519-21525.

Reiländer, H. and Weib, H. M. (1998) Production of G-protein-coupled receptors in yeast Current Opinion Biotechnol. 9, 510-517.

Sander, P., Grümewald, S., Reiländer, H. and Michel, H. (1994) Expression of the human D_{2S} dopamine receptor in the yeasts Saccharomyces cerevisiae and Schizosaccharomyces pombe—a comparative study. FEBS Letts. 344, 4146.

Smits, G. J., Kapteyn, J. C., van den Ende, H. and Klis, F. M. (1999) Cell wall dynamics in yeast. Current Opinion Microbiol. 2, 348-352.

Swift., S., Sheridan, P. J., Covic, L. and Kulipulos, A (2000) PAR1 thrombin receptor-G protein interactions. Separation of binding and coupling determinants in the Gα subunit J. Biol. Chem. 275, 2627-2635.

Vale W, Spiess J, Rivier C, Rivier J. (1981) Characterization of a 41-residue ovine hypothalamic peptide that stimulates secretion of corticotropin and β-endorphin. Science 213, 13941397.

Voyno-Yasenetskaya, T., Conklin, B. R., Gilbert, R. L., Hooley, R, Bourne, H. R. and Barber, D. L. (1994) Gα13 stimulates Na—H exchange. J. Biol. Chem. 269, 47214724.

Watson, P., Davis, K, Didmon, M., Broad, P. and Davey, J. (1999) An RGS protin regulates the pheromone response in tie fission yeast Schizosaccharomyces pombe. Mol. Microbiol. 33,623-634.

1. A Schizosaccharomyces pombe (Sz pombe yeast cell comprising:
(i) a G-Protein Coupled Receptor (GPCR)-regulated signalling pathway which is derepressed during the mitotic phase of cell growth;
(ii) a reporter system for reporting a signal mediated by the GPCR-regulated signalling pathway; and wherein:
(a) the GPCR is heterologous, and/or
(b) the reporter system comprises a reporter gene and a promoter, the reporter gene being operatively linked to the promoter, and the promoter being regulatable by the GPCR, at least one of the reporter gene and the promoter being heterologous.

2. A yeast cell as claimed in claim 1, wherein the GPCR-regulated signalling pathway is derepressed during the mitotic phase of cell growth by disruption of a nutritional control pathway.

3. A yeast cell according to claim 1 or claim 2, wherein the yeast cell is adenylate cyclase deficient.

4. A yeast cell according to any preceding claim which comprises a mutated pat] gene, or in which the endogenous pat1 gene has been deleted.

5. A yeast cell as claimed in any one of claims 1 to 4, wherein the reporter system comprises a reporter gene and a promoter, the reporter gene being operatively linked to the promoter, and the promoter being regulatable by the GPCR, at least one of the reporter gene and the promoter being heterologous.

6. A yeast cell as claimed in claim 5, wherein the reporter gene is heterologous.

7. A yeast cell according to any preceding claim, wherein the GPCR is a mammalian GPCR.

8. A yeast cell according to any preceding claim comprising a heterologous or chimeric G-protein subunit.

9. A yeast cell according to claim 8, wherein the chimeric G-protein subunit is a Gα-transplant.

10. A yeast cell according to claim 9 wherein the Gα-transplant is selected from the following transplants:
Gαq (SEQ ID 17)
Gαs (SEQ ID 16)
Gαo (SEQ ID 18)
Gαi2 (SEQ ID 19)
Gαi3 (SEQ ID 20)
Gαz (SEQ ID 21)
Gα12 (SEQ ID 22)
Gα13 (SEQ ID 23)
Gα14 (SEQ ID 24) and
Gα16 (SEQ ID 25)

11. A yeast cell according to claim 5, wherein the reporter system is regulated by yeast mating pheromone binding to its GPCR

12. A yeast cell according to claim 11, wherein the yeast mating pheromone is P-factor pheromone.

13. A yeast cell according to claim 12, wherein the reporter system-is operatively linked to an sxa2 promoter or a homologue or analogue thereof.

14. A yeast cell according to any preceding claim wherein the reporter system is integrated into the chromosome of the yeast cell.

15. A yeast cell according to any preceding claim, wherein the yeast cell has a stable mating type.

16. A yeast cell according to any preceding claim wherein the yeast cell is rgs1 deficient.

17. A yeast cell according to any preceding claim, wherein the yeast cell is pmp1 deficient.

18. A yeast cell according to any preceding claim, wherein the yeast cell is sxa2 deficient.

19. A yeast cell according to claim 18, wherein at least a part of the endogenous sxa2 gene has been deleted.

20. A yeast cell according to claim 19, wherein the reporter gene replaces the deleted mra2 gene.

21. A yeast cell according to any preceding claim wherein the reporter gene encodes orotidine-5′-phosphate decarboxylase (the product of the Sz. pombe ura4 gene), β-galactosidase (the product of the bacterial lacZ gene), β-lactamase, aequorin, green fluorescent protein or luciferase.

22. A yeast cell which is Schizosaccharomyces pombe strain JY546 deposited as accession number NCYC2984.

23. A yeast cell according to any preceding claim additionally comprising one or more compounds, to be assayed for their effect on GPCR-regulated expression of the reporter system, or a DNA molecule encoding one or more peptides or proteins to be assayed

24. A yeast cell according to claim 23, comprising one or more plasmids encoding the or each peptide or protein.

25. A yeast cell according to claim 24, wherein the DNA encoding peptide or protein is transcribed under the control of a thiamine-regulated nmt1 promoter.

26. A yeast cell according to claim 23 or claim 24, wherein the peptide or protein is of a random sequence.

27. A yeast cell according to any preceding claim, wherein the yeast cell expresses an orphan receptor as the GPCR and the reporter system is regulatable by the orphan receptor.

28. An isolated nucleic acid molecule comprising an sxa2 promoter, or a homologue or analogue thereof, operatively linked to an exogenous reporter gene.

29. An isolated nucleic acid molecule according to claim 28, wherein the reporter system encodes orotidine-5′-phosphate decarboxylase (the product of the Sz. pombe ura4 gene), β-galactosidase (the product of the bacterial lacZ gene), a β-lactamase, aequorin, green fluorescent protein or luciferase.

30. An isolated nucleic acid molecule encoding a Gα-transplant having a nucleic acid sequence selected from:
Gαq (SEQ ID 17)
Gαs (SEQ ID 16)
Gαo (SEQ ID 18)
Gαi2 (SEQ ID 19)
Gαi3 (SEQ ID 20)
Gαz (SEQ ID 21)
Gα12 (SEQ ID 22)
Gα13 (SEQ ID 23)
Gα14 (SEQ ID 24) and
Gα16 (SEQ ID 25);
or which differs from the one or more of the sequences due to degeneracy in the genetic code.

31. Use of:
(a) a Schizosaccharomyces pombe (Sz. pombe) yeast cell comprising:
(i) a G-Protein Coupled Receptor (GPCR)-regulated signalling pathway which is derepressed during the mitotic phase of cell growth;
(ii) a reporter system for reporting a signal mediated by the GPCR-regulated signalling pathway; or;
(b) an isolated nucleic acid molecule according to any one of claims 28 to 30 to study GPCR-regulated activity.

32. An assay comprising the use of a yeast cell or isolated DNA molecule according to any one of claims to 31.

33. A method of determining the effect of a compound, on GPCR-regulated activity comprising the steps of
(i) providing a Schizosaccharomyces pombe (Sz. pombe) yeast cell comprising:
(a) a G-Protein Coupled Receptor (GPCR)regulated signalling pathway which is derepressed during the mitotic phase of cell growth;
(b) a reporter system for reporting a signal mediated by the GPCR-regulated signalling pathway;
(ii) introducing the compound, to the yeast cell; and
(iii) noting the output of the reporter system.

34. Use of a yeast cell according to claim 27 to identify compounds, which affect the ability of the orphan GPCR to regulate the reporter system.

35. Use of a Schizosaccharomyces pombe (Sz. pombe) yeast cell comprising:
(i) a G-Protein Coupled Receptor (GPCR)-regulated signalling pathway which is derepressed during the mitotic phase of cell growth;
(ii) a reporter system for reporting a signal mediated by the GPCR-regulated signalling pathway;
to identify a regulator or a mutant of a component of a GPCR-regulated pathway.

36. A method of identifying a reagent that modulates GPCR-regulated signalling pathways comprising:
(i) providing a Schizosaccharomyces pombe (Sz. pombe) yeast cell comprising:
(a) a G-Protein Coupled Receptor (GPCR)-regulated si pathway which is derepressed during the mitotic phase of cell growth;
(b) a reporter system for reporting a signal mediated by the GPCR-regulated signaling pathway,
(ii) producing a random peptide within the yeast cell; and
(iii) measuring an amount of reporter activity produced.

37. A compound capable of modulating GPCR activity identified by a method according to claim 33 or claim 36.

38. A method of determining whether a G-Protein Coupled Receptor (GPCR) is coupled to a cell signalling pathway, the method comprising comparing the ligand-independent reporter system output of a Schizosaccharomyces pombe (Sz. pombe) yeast cell comprising:
(i) a G-Protein Coupled Receptor (GPCR)-regulated signalling pathway which is derepressed during the mitotic phase of cell growth;
(ii) a reporter system for reporting a signal mediated by the GPCR-regulated signaling pathway;
with the reporter system output of a reference cell which lacks a functional GPCR

39. A Schizosaccharomyces pombe (Sz. pombe) yeast cell comprising:
(i) a G-Protein Coupled Receptor (GPCR)-regulated signalling pathway which is derepressed during the mitotic phase of cell growth, wherein the GPCR is absent or otherwise rendered non-functional;
(ii) a reporter system for reporting a signal mediated by the GPCR-regulated signing pathway.

40. An assay kit comprising a yeast cell or isolated nucleic acid molecule as defined in any one of claims 1 to 30 and 39.
